(19) United States
(12) Patent Application Publication
(10) Pub. No.: US 2025/0271439 A1
(43) Pub. Date: Aug. 28, 2025

(54) PROTEOMIC SIGNATURE OF PLASMA EXTRACELLULAR VESICLES CLASSIFIES RESPONSE TO DOXORUBICIN AND CANCER

(71) Applicant: Washington University, St. Louis, MO (US)

(72) Inventors: Jason Held, St. Louis, MO (US); Brian Van Tine, St. Louis, MO (US)

(21) Appl. No.: 19/205,569

(22) Filed: May 12, 2025

Related U.S. Application Data
(60) Provisional application No. 63/345,475, filed on May 25, 2022.

(51) Int. Cl. G01N 33/574; G01N 33/573

(52) U.S. Cl. CPC G01N 33/57488; G01N 33/573; G01N 33/57496; G01N 2333/90661; G01N 2333/978; G01N 2570/00

(57) ABSTRACT

The present disclosure relates to methods of identifying a subject as a candidate for cancer treatment and methods of treating cancer in a subject in need thereof according to the detection of plasma extracellular vesicles and/or plasma extracellular vesicle proteins. In addition, the present disclosure relates to kits comprising reagents and substrates for identifying a subject as a candidate for cancer treatment.

### REFERENCE TO RELATED APPLICATIONS

This application claims the benefit under 35 U.S.C. § 119(e) of U.S. Provisional Patent Application No. 63/645,475, filed May 10, 2024, the entire disclosure of which is incorporated herein by reference.

### FIELD OF THE INVENTION

The present disclosure generally relates to plasma extracellular vesicles and methods of detecting and/or treating cancer.

### BACKGROUND OF THE INVENTION

Extracellular vesicles (EVs) are a heterogenous group of small, membranous vesicles released by cells into the extracellular environment that play a role in intercellular communication, cell signaling, and disease progression. EVs present in plasma have emerged as a promising source of biomarkers due to their stability and abundance, and a liquid biopsy of plasma EVs is minimally invasive and serially obtainable. Protein biomarkers identified in plasma exosomes have shown excellent potential in cancer diagnosis and prognosis (Hoshino et al., Cell, 2020, 182, 1044-1061.e18; Vinik et al., Sci Adv 6, 2020, eaba5714; and Whiteside, Nature Reviews Clinical Oncology, 2020, 17, 179-720).

Sarcoma is a rare type of cancer that arises from connective tissues such as bone, muscle, and cartilage, accounting for approximately 1% of all adult cancers and 15-20% of pediatric cancers. Despite advances in treatment, the prognosis for sarcoma patients remains poor, with a high risk of metastasis and relapse (Hoshino et al., Cell, 2020, 182, 1044-1061.e18). Therefore, the identification of new biomarkers for early diagnosis, prognosis, and monitoring of sarcoma is essential to improve patient outcomes. Doxorubicin is a commonly used chemotherapy drug for sarcoma treatment which often causes adverse effects, especially cardiotoxicity. Accordingly, it is crucial to monitor the efficacy and safety of doxorubicin treatment in sarcoma patients. A noninvasive biomarker identifying which patients would have a poor response to doxorubicin would be beneficial in the clinic to reduce unnecessary treatment and toxicity.

To identify proteins in plasma EVs that differentiate sarcoma patients from healthy volunteers, as well as patients that responded to doxorubicin chemotherapy, samples were obtained from the doxorubicin monotherapy control arm of the Phase 3 SARC021 clinical trial (TH CR-406/SARC021). A streamlined and robust plasma EV proteomic workflow coupled with a machine learning approach led to the development of proteomic signatures of sarcoma and doxorubicin sensitivity (FIG. 1A). The present disclosure is directed to protein biomarkers in plasma EVs which provide a valuable tool for diagnosing, monitoring, and treating sarcoma and cancer patients, including development of proteomic signatures suitable for unbiased classification of cancers and doxorubicin treatment with high accuracy.

### SUMMARY OF THE INVENTION

Among the various aspects of the present disclosure is the provision of plasma extracellular vesicles and methods of detecting and/or treating cancer.

Briefly, therefore, the present disclosure is directed to a method for identifying a human subject as a candidate for treating cancer that involves determining whether or not a sample from the subject comprises a plasma extracellular vesicle and/or a plasma extracellular vesicle protein and identifying the subject as a candidate for treating cancer by administering an anticancer treatment wherein the anticancer treatment is not administering doxorubicin when the plasma extracellular vesicle and/or the plasma extracellular vesicle protein is detected. The plasma extracellular vesicle and/or plasma extracellular vesicle protein sample is subjected to a detection assay suitable for detecting: i) a plasma extracellular vesicle and/or ii) a plasma extracellular vesicle protein, wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine--tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

Another aspect of the present disclosure is directed to a method for treating a human subject having cancer that involves performing or having performed an assay on a biological sample from the subject to identify the subject as having: i) a plasma extracellular vesicle and/or ii) a plasma extracellular vesicle protein and administering an anticancer treatment wherein the anticancer treatment is not administering doxorubicin to the subject having the plasma extracellular vesicle and/or plasma extracellular vesicle protein wherein the presence of the plasma extracellular vesicle and/or the plasma extracellular vesicle protein indicates that the subject is a candidate for treating the cancer by administering an anticancer treatment wherein the anticancer treatment is not administering doxorubicin. The plasma extracellular vesicle and/or plasma extracellular vesicle protein sample is subjected to a detection assay suitable for detecting: i) a plasma extracellular vesicle and/or ii) a plasma extracellular vesicle protein, wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

Another aspect of the present disclosure is directed to a kit suitable for identifying a human subject as a candidate for treating cancer. The kit includes reagents and/or substrates suitable for detecting a plasma extracellular vesicle and/or a plasma extracellular vesicle protein, wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

Another aspect of the present disclosure is directed to a method of determining a treatment regimen for a subject having a tumor. The method involves obtaining, from the subject having the tumor, a biopsy of tumor tissue and a biopsy of tissue adjacent to the tumor, and separating plasma extracellular vesicles and/or plasma extracellular vesicle proteins from the obtained samples. Plasma extracellular vesicle protein from the separated plasma extracellular vesicle is isolated to form plasma extracellular vesicle and plasma extracellular vesicle protein samples, and the plasma extracellular vesicle and plasma extracellular vesicle protein samples are subjected to a detection assay suitable for detecting proteins differentially expressed in the tumor tissue versus adjacent, non-tumor tissue. A treatment regimen for the subject is identified based on said subjecting.

Another aspect of the present disclosure is directed to a method of identifying drug targets for cancer therapy. The method involves obtaining, from each of a plurality of subjects having a particular tumor, a biopsy of tumor tissue and a biopsy of tissue adjacent to said tumor, and separating plasma extracellular vesicles and plasma extracellular vesicle protein from the obtained samples. Plasma extracellular vesicle protein from the separated extracellular vesicle is isolated to form extracellular vesicle and plasma extracellular vesicle protein samples, and the plasma extracellular vesicle and plasma extracellular vesicle protein samples is subjected to proteomic analysis to identify proteins differentially expressed in the tumor tissue versus tissue adjacent said tumor. Drug targets for cancer therapy are identified based on said subjecting.

Another aspect of the present disclosure is directed to a method of treating a subject having cancer. The method involves selecting a subject having a tumor, wherein plasma extracellular vesicles from tumor tissue express phosphorylase b kinase regulatory subunit alpha, liver isoform (PHKA2) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express integrin alpha-5 (ITGA5), sphingolipid delta(4)-desaturase DES (DEGS1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express E3 ubiquitin/ISG15 ligase (TRIM25) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express conserved oligomeric Golgi complex subunit 3 (COG3) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express rab3 GTPase-activating protein non-catalytic subunit (RAB3GAP2) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express mannosyl-oligosaccharide glucosidase (MOGS) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express ER membrane protein complex subunit 10 (EMC10) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express bifunctional purine biosynthesis protein (ATIC) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express ribose-5-phosphate isomerase (RPIA) and administering to said subject an anticance treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express sterol-4-alpha-carboxylate 3-dehydrogenase and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express decarboxylating (NSDHL) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express dehydrogenase/reductase SDR family member 7B (DHRS7B) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express LIM domain-containing protein 1 (LIMD1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express thymidine kinase 2, mitochondrial (TK2) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express isovaleryl-CoA dehydrogenase, mitochondrial (IVD) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express NIF3-like protein 1 (NIF3L1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express ubiquitin carboxyl-terminal hydrolase 15 (USP15) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express asparagine—tRNA ligase, cytoplasmic (NARS1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express immunoglobulin lambda variable 2-11 (IGLV2-11) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express semenogelin-1 (SEMG1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express immunoglobulin kappa variable 1-13 (IGKV1-13) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express acylamino-acid-releasing enzyme (APEH) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express aldo-keto reductase family 1 member A1 (AKR1A1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express sodium/potassium-transporting ATPase subunit beta-3 (ATP1B3) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express F-actin-uncapping protein LRRC16A (CARMIL1) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express nuclear migration protein (NUDC) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express sorting nexin-2 (SNX2) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express immunoglobulin lambda variable 4-69 (IGLV4-69) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express phosphatidylinositol 4-phosphate 5-kinase type-1 gamma (PIP5K1C) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express supervillain (SVIL) and administering to said subject an anticancer treatment wherein the anticancer treatment is not administering doxorubicin.

Another aspect of the present disclosure is directed to a method of treating a subject having cancer. The method involves selecting a subject having a tumor, wherein plasma extracellular vesicles from tumor tissue express prelamin-A/C (LMNA) and administering to said subject an anticancer treatment wherein the anticancer treatment is not administering doxorubicin.

To identify new candidate markers for cancer, proteomic profiling of plasma extracellular vesicles in two distinct cohorts of cancer patients coupled with machine learning-based classification was established. The first cohort included 40 sarcoma patients treated with doxorubicin, the standard of care therapy that has a high risk of cardiotoxicity. Sarcoma had a widespread effect on the plasma proteome, significantly altering the abundance of 44% of robustly detected proteins and enabling perfect classification of cancer and normal patients. Validation of these results in an independent cohort of cancer patients and healthy volunteers that utilized different EV enrichment and proteomic technologies generated an atlas of 837 pan-cancer-regulated plasma EV proteins in an integrated dataset of 122 cancer patients, 23m cancer types, and 55 healthy volunteer controls, the largest global plasma EV proteomics dataset to date. A proteomic signature was developed that could classify cancer patients from healthy volunteers with a mean ROC AUC of 0.98, specificity of 96%, diagnostic odds ratio of 328, and balanced accuracy of 95%. The plasma EV proteome of sarcoma patients prior to doxorubicin treatment also differentiated responders versus non-responders, and a proteomic signature was developed that could classify responders with a mean accuracy of 70.8%, AUROC of 0.71, sensitivity of 81.0% and odds ratio of 6.6. Taken together, the proteome of plasma EVs in cancer patients is distinctive and can provide pre-treatment indications of anticancer treatment response which could be especially useful for drugs with high toxicity such as doxorubicin.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

The patent or application file contains at least one drawing executed in color. Copies of this patent or patent application publication with color drawing(s) will be provided by the Office upon request and payment of the necessary fee.

FIG. 1A shows plasma was isolated from healthy volunteers, or sarcoma patients in the SARC021 clinical trial before and after treatment with 75 mg/m² doxorubicin at baseline and after 2 cycles. EVs were captured using the Vn96 capture technology followed by label free quantitative proteomics, data analysis, and statistics.

FIG. 1B shows size analysis of a representative plasma EV sample.

FIG. 1C shows mean LC-MS peak area of all proteins detected across the entire data set, with conventional EV marker proteins highlighted in red.

FIG. 1D shows summed LC-MS ion intensity quantitation of each sample for all proteins identified (black) and the 10 most abundant EV proteins in the ExoCarta database²⁸ (red).

FIG. 2A shows hierarchical clustering of each sample based on protein expression of plasma EVs.

FIG. 2B shows multidimensional scaling coordinates colored by sarcoma subtype with shapes and ellipses grouping sarcoma patients versus healthy volunteers.

FIG. 2C shows a volcano plot and example proteins that are differentially expressed in healthy volunteers versus sarcoma patients prior to doxorubicin treatment. The adjP-value is Benjamini-Hochberg corrected.

FIG. 2D shows example proteins that are differentially expressed in healthy volunteers versus sarcoma patients prior to doxorubicin treatment. The adjP-value is Benjamini-Hochberg corrected.

FIG. 2E shows accuracy and AUROC of the KNN algorithm to classify sarcoma patients from healthy volunteers using holdout testing across 7 randomized splits of the samples into independent training, validation, and testing sets.

FIG. 2F shows AUROC of the KNN algorithm to classify sarcoma patients from healthy volunteers using holdout testing across 7 randomized splits of the samples into independent training, validation, and testing sets.

FIG. 3A shows pan-cancer plasma EV protein discovery workflow that integrates the sarcoma cohort of this study with the cancer plasma EV proteomics reported by Hoshino et al¹. 23 total cancer types include angiosarcoma, breast, colorectal, ependymoma, epithelioid sarcoma, germinoma, leiomyosarcoma, liposarcoma, lung, lymphoma, malignant peripheral nerve sheath tumor (MPNST), melanoma, mesothelioma, myxofibrosarcoma, neuroblastoma, osteosarcoma, pancreatic cancer, stomach, undifferentiated pleomorphic sarcoma/malignant fibrous histiocytoma, and wilms. q indicates a Benjamini-Hochberg corrected p-value and n.s. indicates not statistically significant. Pathway analysis was performed with WebGestalt⁹⁵.

FIG. 3B shows detection of pan-cancer EV proteins across cancer types and individual datasets as well as their AUROC for the pan-cancer dataset.

FIG. 3C shows a machine learning pipeline.

FIG. 3D shows average accuracy, performance, and AUROC classifying healthy volunteers versus cancer patients on the 7 held out Test sets using the plasma EV proteome and the KNN classifier.

FIG. 3E shows AUROC classifying healthy volunteers versus cancer patients on the 7 held out Test sets using the plasma EV proteome and the KNN classifier.

FIG. 4A shows correlation matrix of plasma EV proteomes across cancer types.

FIG. 4B shows unsupervised PCA and supervised T-SNE classification of the plasma EV proteome for cancers with at least 4 samples in the pan-cancer dataset. A 95% confidence interval (C.I.) of all sarcoma samples in the pan-cancer dataset is shown.

FIG. 4C shows supervised T-SNE classification of the plasma EV proteome for cancers with at least 4 samples in the pan-cancer dataset. A 95% confidence interval (C.I.) of all sarcoma samples in the pan-cancer dataset is shown.

FIG. 4D shows detection of P4HB in MPNST samples and healthy volunteers for the pan-cancer dataset.

FIG. 5A shows a volcano plot of fold change in EV protein expression before versus after doxorubicin treatment in drug sensitive or resistant sarcoma patients.

FIG. 5B shows a volcano plot of fold change in EV protein expression in doxorubicin-sensitive versus-resistant sarcoma patients.

FIG. 5C shows supervised LDA and coefficients of healthy volunteers and sarcoma patients prior to treatment.

FIG. 5D shows ROC curves and protein expression and ROC in sarcoma patients for individual proteins in plasma EVs.

FIG. 5E shows protein expression and ROC in sarcoma patients for individual proteins in plasma EVs.

FIG. 5F shows classification accuracy, performance, and AUROC of sarcoma patient doxorubicin sensitivity prior to treatment using the plasma EV proteome and the XGB classifier.

### DETAILED DESCRIPTION OF THE INVENTION

The present disclosure is directed to methods of identifying, diagnosing, characterizing, and treating cancer conditions, or lack thereof, in a subject based on plasma extracellular vesicle and/or plasma extracellular vesicle protein derived and tissue derived plasma extracellular vesicle and/or plasma extracellular vesicle protein signatures. These methods involve obtaining a liquid biopsy sample and/or a tissue sample from a subject, separating from these samples plasma extracellular vesicles and plasma extracellular vesicle protein, isolating plasma extracellular vesicle protein from the separated plasma extracellular vesicle and detecting the presence and/or absence of the plasma extracellular vesicle proteins as described herein. Plasma extracellular vesicle proteins of the plasma extracellular vesicle protein signatures described herein are referred to interchangeably by their protein name and gene name.

A first aspect of the present disclosure is directed to a method for identifying a human subject as a candidate for treating cancer that involves determining whether or not a sample from the subject comprises a plasma extracellular vesicle and/or a plasma extracellular vesicle protein and identifying the subject as a candidate for treating cancer by administering an anticancer treatment wherein the anticancer treatment is not administering doxorubicin when the plasma extracellular vesicle and/or the plasma extracellular vesicle protein is detected. The plasma extracellular vesicle and/or plasma extracellular vesicle protein sample is subjected to a detection assay suitable for detecting: i) a plasma extracellular vesicle and/or ii) a plasma extracellular vesicle protein, wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit

(RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof. In accordance with this method, detecting the presence of one or more plasma extracellular vesicles from (i) is indicative of the presence of cancer in the subject and detecting the presence of one or more plasma extracellular vesicle proteins from (ii) is indicative of the presence of cancer in the subject. In certain embodiments, at least two plasma extracellular vesicle proteins of (ii) are detected.

Another aspect of the present disclosure is directed to a method for screening a subject for the presence of cancer that involves obtaining a liquid biopsy sample from a subject. Plasma extracellular vesicles and plasma extracellular vesicle proteins are separated from the sample, and plasma extracellular vesicle proteins are separated from the plasma extracellular vesicles and isolated to form a plasma extracellular vesicle protein sample. The plasma extracellular vesicle protein sample is subjected to a detection assay suitable for detecting wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine--tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof, thereby detecting the presence or absence of the plasma extracellular vesicle protein in the plasma extracellular vesicle protein sample. In accordance with this method, detecting the presence of one or more plasma extracellular vesicle proteins is indicative of the presence of cancer in the subject.

In one embodiment, the plasma extracellular vesicle protein that is detected is phosphorylase b kinase regulatory subunit alpha, liver isoform (PHKA2). In one embodiment, the plasma extracellular vesicle protein that is detected is integrin alpha-5 (ITGA5). In one embodiment, the plasma extracellular vesicle protein that is detected is sphingolipid delta(4)-desaturase DES (DEGS1). In one embodiment, the plasma extracellular vesicle protein that is detected is E3 ubiquitin/ISG15 ligase (TRIM25). In one embodiment, the plasma extracellular vesicle protein that is detected is conserved oligomeric Golgi complex subunit 3 (COG3). In one embodiment, the plasma extracellular vesicle protein that is detected is rab3 GTPase-activating protein non-catalytic subunit (RAB3GAP2). In one embodiment, the plasma extracellular vesicle protein that is detected is mannosyl-oligosaccharide glucosidase (MOGS). In one embodiment, the plasma extracellular vesicle protein that is detected is ER membrane protein complex subunit 10 (EMC10). In one embodiment, the plasma extracellular vesicle protein that is detected is bifunctional purine biosynthesis protein (ATIC). In one embodiment, the plasma extracellular vesicle protein that is detected is ribose-5-phosphate isomerase (RPIA), sterol-4-alpha-carboxylate 3-dehydrogenase, decarboxylating (NSDHL). In one embodiment, the plasma extracellular vesicle protein that is detected is dehydrogenase/reductase SDR family member 7B (DHRS7B). In one embodiment, the plasma extracellular vesicle protein that is detected is LIM domain-containing protein 1 (LIMD1). In one embodiment, the plasma extracellular vesicle protein that is detected is thymidine kinase 2, mitochondrial (TK2). In one embodiment, the plasma extracellular vesicle protein that is detected is isovaleryl-CoA dehydrogenase, mitochondrial (IVD). In one embodiment, the plasma extracellular vesicle protein that is detected is NIF3-like protein 1 (NIF3L1). In one embodiment, the plasma extracellular vesicle protein that is detected is ubiquitin carboxyl-terminal hydrolase 15 (USP15). In one embodiment, the plasma extracellular vesicle protein that is detected is asparagine—tRNA ligase, cytoplasmic (NARS1). In one embodiment, the plasma extracellular vesicle protein that is detected is immunoglobulin lambda variable 2-11 (IGLV2-11). In one embodiment, the plasma extracellular vesicle protein that is detected is semenogelin-1 (SEMG1). In one embodiment, the plasma extracellular vesicle protein that is detected is immunoglobulin kappa variable 1-13 (IGKV1-13). In one embodiment, the plasma extracellular vesicle protein that is detected is acylamino-acid-releasing enzyme (APEH). In one embodiment, the plasma extracellular vesicle protein that is detected is aldo-keto reductase family 1 member A1 (AKR1A1). In one embodiment, the plasma extracellular vesicle protein that is detected is sodium/potassium-transporting ATPase subunit beta-3 (ATP1B3). In one embodiment, the plasma extracellular vesicle protein that is detected is F-actin-uncapping protein LRRC16A (CARMIL1). In one embodiment, the plasma extracellular vesicle protein that is detected is nuclear migration protein (NUDC). In one embodiment, the plasma extracellular vesicle protein that is detected is sorting nexin-2 (SNX2). In one embodiment, the plasma extracellular vesicle protein that is detected is immunoglobulin lambda variable 4-69 (IGLV4-69). In one embodiment, the plasma extracellular vesicle protein that is detected is phosphatidylinositol 4-phosphate 5-kinase type-1 gamma (PIP5K1C). In one embodiment, the plasma extracellular vesicle protein that is detected is supervillain (SVIL). In one embodiment, the plasma extracellular vesicle protein that is detected is prelamin-A/C (LMNA).

In accordance with this aspect of the present disclosure, these methods are employed to screen a subject for the general presence of cancer based on the presence and/or absence of the described proteins in the plasma extracellular vesicle and plasma extracellular vesicle protein sample. For example, these methods can be employed during a regularly scheduled physical examination to achieve early detection of cancer in the subject. Alternatively, these methods may be employed in a subject possessing a tumor or abnormal tissue mass, where it is unknown if the tumor or tissue mass is benign or malignant. Accordingly, when either method is employed to detect the general presence of cancer in a subject, the presence of one or more plasma extracellular vesicles from (i) is indicative of the presence of cancer in the subject and detecting the presence of one or more plasma extracellular vesicle proteins from (ii) is indicative of the presence of cancer in the subject.

In some embodiments, at least one, at least two, at least three, at least four, at least 5, at least 6, at least 7, at least 8, at least 9, at least 10 or great than 10 plasma extracellular vesicle proteins from the proteins of group (ii) are subject to detection, and the detection of any one or more of these proteins in the sample indicates the presence of cancer in the subject.

When utilized together, the detection of one or more plasma extracellular vesicles of (i) and/or the detection of one or more plasma extracellular vesicle proteins of (ii) is indicative of the presence of cancer in the subject or the presence of a malignant tumor in the subject. Alternatively, detecting the absence of one or more plasma extracellular vesicles of (i) and/or the absence of one or more plasma extracellular vesicle proteins of (ii) is indicative that the subject does not have cancer or that any tumor or tissue mass is a benign tumor or tissue mass. Detecting both the presence and/or absence of tumor-associated and non-tumor associated plasma extracellular vesicles and/or plasma extracellular vesicle proteins significantly improves the diagnostic integrity of the methods described herein.

The methods described herein can be used as a diagnostic approach before more invasive testing (e.g., liquid biopsy prior to tissue biopsy) or it may follow another diagnostic approach (e.g., liquid biopsy to detect biomarkers after mammogram, ultrasound, MRI, tissue biopsy, PSA blood test, or genetic testing) to provide additional information or clarify unclear results. Alternatively, the methods may be used as a standard test during a yearly doctor's visit to generally detect the presence or absence of cancer in a subject. Accordingly, the subject tested using the methods disclosed herein may have one or more risk factors for a cancer and be asymptomatic. The subject may be asymptomatic of a cancer. The subject may have one or more risk factors for a cancer. The subject may be symptomatic for a cancer and have one or more risk factors of the cancer. The subject may have or be suspected of having a cancer or a tumor. The subject may have a tumor, and the status of the tumor, e.g., benign or malignant, is unknown. The subject may be a patient being treated for a cancer. The subject may be predisposed to a risk of developing a cancer or a tumor. The subject may be in remission from a cancer or a tumor. The subject may not have a cancer, may not have a tumor, or may not have a cancer or a tumor. The subject may be healthy.

Another aspect of the present disclosure is directed to a method for screening a subject for the presence of cancer that involves obtaining a liquid biopsy sample from a subject. Plasma extracellular vesicles and plasma extracellular vesicle proteins are separated from the sample, and isolated to form a plasma extracellular vesicle and plasma extracellular vesicle protein sample. The plasma extracellular vesicle and plasma extracellular vesicle protein sample is subjected to a detection assay suitable for detecting any one or more of the proteins selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

In accordance with all aspects of the present disclosure, a “subject” as referred to herein encompasses any animal, but preferably a mammal, e.g., human, non-human primate, a dog, a cat, a horse, a cow, or a rodent. More preferably, the subject is a human. In any embodiment of the present disclosure, the subject has a tumor or tissue mass, where the status of the tumor or mass (i.e., benign or malignant) is unknown. In any embodiment of the present disclosure, the subject has cancer, for example and without limitation, lung cancer, pancreatic cancer, neuroblastoma, osteosarcoma, breast cancer, colorectal cancer, and mesothelioma. In any embodiment, the cancer is a primary tumor, while in other embodiments, the cancer is a secondary or metastatic tumor. In any embodiment, the cancer involves of a tumor of unknown origin.

“Plasma extracellular vesicles and plasma extracellular vesicle proteins” refers to any one or more of the subpopulations of exosomes (i.e., Exo-S and Exo-L) and exomeres. Generally, exosomes are microvesicles released from a variety of different cells, including cancer cells (i.e., “cancer-derived exosomes”). These small vesicles derive from large multivesicular endosomes and are secreted into the extracellular milieu. The precise mechanisms of exosome release/shedding remain unclear; however, this release is an energy-requiring phenomenon, modulated by extracellular signals. They appear to form by invagination and budding from the limiting membrane of late endosomes, resulting in vesicles that contain cytosol and that expose the extracellular domain of membrane-bound cellular proteins on their surface. Using electron microscopy, studies have shown fusion profiles of multivesicular endosomes with the plasma membrane, leading to the secretion of the internal vesicles into the extracellular environment. The rate of exosome release is significantly increased in most neoplastic cells and occurs continuously. Increased release of exosomes and their accumulation appear to be important in the malignant transformation process.

In accordance with the methods of the present disclosure, for the purposes of a liquid biopsy, plasma extracellular vesicles and plasma extracellular vesicle proteins can be isolated or obtained from most biological fluids including, without limitation, whole blood, blood serum, blood plasma, ascites fluid, cyst fluid, pleural fluid, peritoneal fluid, cerebrospinal fluid, tears, urine, saliva, sputum, nipple aspirates, lymph fluid, synovial fluid, amniotic fluid, semen, follicular fluid, fluid of the respiratory, intestinal, and genitourinary trances, breast milk, intra-organ system fluid, conditioned media from tissue explant culture, or combinations thereof.

Another aspect of the present disclosure is directed to a method for treating a human subject having cancer that involves performing or having performed an assay on a biological sample from the subject to identify if the subject has having: i) a plasma extracellular vesicle and/or ii) a plasma extracellular vesicle protein and administering an anticancer treatment wherein the anticancer treatment is not administering doxorubicin to the subject having the plasma extracellular vesicle and/or plasma extracellular vesicle protein wherein the presence of the plasma extracellular vesicle and/or the plasma extracellular vesicle protein indicates that the subject is a candidate for treating the cancer by administering an anticancer treatment wherein the anticancer treatment is not administering doxorubicin. The plasma extracellular vesicle and/or plasma extracellular vesicle protein sample is subjected to a detection assay suitable for detecting: i) a plasma extracellular vesicle and/or ii) a plasma extracellular vesicle protein, wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

The term “treatment” refers to administration of a therapy to a patient having a tumor, where the therapy is administered in a manner effective to inhibit growth of the tumor or inhibit or prevent metastasis from occurring. Treatment as used herein also encompasses treatment that is effective to delay, slow, or lessen the severity of a primary tumor or metastasis.

Suitable anticancer therapies for treating cancer are known in the art, and include, for example and without limitation, chemotherapy, including, without limitation, anthracyclines, such as epirubicin (Ellence), taxanes, such as paclitaxel (Taxol) and docetaxel (Taxotere), 5-fluorouracil (5-FU), capecitabine, cyclophosphamide (Cytoxan), carboplatin (Paraplatin), albumin-bound paclitaxel (Abraxane), platinum agents (cisplatin, carboplatin), vinorelbine (Navelbine), capecitabine (Xeloda), gemcitabine (Gemzar), ixabepilone (Ixempra), eribulin (Halaven); hormone therapy, including, without limitation, tamoxifen, toremifene (Fareston), fulvestrant (faslodex); aromatase inhibitors (e.g., Letrozole (Femara), Anastrozole (Arimidex), and Exemestane (Aromasin)); targeted therapeutics, including, without limitation, monoclonal antibody therapeutics (e.g., HER2 antibody (Trastuzumab (Herceptin)), Pertuzumab (Perjeta), Margetuximab (Margenza), antibody-drug conjugates (e.g., Ado-trastuzumab emtansine (Kadcyla or TDM-1), Fam-trastuzumab deruxtecan (Enhertu), Sacituzumab govitecan (Trodelvy)), kinase inhibitors (e.g., Lapatinib (Tykerb), Neratinib (Nerlynx), Tucatinib (Tukysa)), CDK4/6 inhibitors (e.g., Palbociclib (Ibrance), ribociclib (Kisqali), and abemaciclib (Verzenio)), mTOR inhibitors (e.g., Everolimus (Afinitor)), PI3K inhibitor (e.g., Alpelisib (Piqray)), PARP inhibitors (e.g., Olaparib (Lynparza) and talazoparib (Talzenna)); and immunotherapy, including, without limitation, immune checkpoint inhibitors, e.g., PD-1 inhibitors (Pembrolizumab and Atezolizumab (Tecentriq)).

In practicing the methods of the present disclosure, the administering step is carried out to achieve treatment of the identified tumor. Such administration can be carried out systemically or via direct or local administration to the primary tumor site. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterially, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. By way of example, intra-ommaya and intrathecal administration are suitable modes for direct administration into the brain for existing metastases. The mode of affecting delivery of agent will vary depending on the type of prophylactic agent (e.g., an antibody or small molecule).

The therapeutic drug may be orally administered, for example, with an inert diluent, or with an assimilable edible carrier, or it may be enclosed in hard or soft shell capsules, or it may be compressed into tablets, or they may be incorporated directly with the food of the diet. Therapeutic drugs may also be administered in a time release manner incorporated within such devices as time-release capsules or nanotubes. Such devices afford flexibility relative to time and dosage. For oral therapeutic administration, the agents may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit.

When the treatment is administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations of the therapeutic drug suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In addition to the formulations described previously, the therapeutic drug may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the therapeutic drug vary depending upon many different factors, including type and stage of the primary cancer, means of administration, target site, physiological state of the patient, other medications or therapies administered, and physical state of the patient relative to other medical complications. Treatment dosages need to be titrated to optimize safety and efficacy.

In accordance with all aspects and embodiments described herein where plasma extracellular vesicles and plasma extracellular vesicle proteins are separated or isolated from a biological tissue or fluid sample, this separation and/or isolation can be performed using a method that involves contacting the biological tissue or fluid sample with one or more binding molecules specific for the thirteen exosomal protein markers identified herein. As disclosed herein, thirteen universal protein exosomal markers have been identified to improve the isolation of human exosomes from biological samples. The thirteen identified exosomal markers include alpha-2-macroglobulin, beta-2-Microglobulin, stomatin, filamin A, fibronectin 1, gelsolin, hemoglobin subunit Beta, galectin-3-binding protein, ras-related protein 1b, actin beta, joining chain of multimeric IgA and IgM, peroxiredoxin-2, and moesin. The biological sample is thus contacted with the one or more binding molecules specific for the aforementioned exosomal markers under conditions suitable for the one or more binding molecules to bind its respective exosomal marker protein in the sample to form one or more binding molecule-target protein complexes. The one or more binding molecule-target protein complexes are selected for, thereby separating the extracellular vesicle and particles from the biological sample. Binding molecules capable of binding an exosomal marker proteins can be used alone or in combination to isolate or separate exosome from a sample or to enrich the purity of a previously fractionated biological sample.

In certain embodiments, the sample is contacted with at least two different binding molecules or with at least three different binding molecules to enhance the isolation of extracellular vesicles and particles from a biological liquid or tissue sample.

An enriched population of extracellular vesicles and particles can also be obtained from a biological sample using other methods known in the art (see e.g., WO2019/109077 to Lyden et al., which is hereby incorporated by reference in its entirety). For example, exosomes may be concentrated or isolated from a biological sample using size exclusion chromatography, density gradient centrifugation, differential centrifugation (Raposo et al. “B lymphocytes Secrete Antigen-presenting Vesicles,” J Exp Med 183(3): 1161-72 (1996), which is hereby incorporated by reference in its entirety), anion exchange and/or gel permeation chromatography (for example, as described in U.S. Pat. No. 6,899,863 to Dhellin et al., and U.S. Pat. No. 6,812,023 to Lamparski et al., which are hereby incorporated by reference in their entirety), sucrose density gradients or organelle electrophoresis (for example, as described in U.S. Pat. No. 7,198,923), magnetic activated cell sorting (MACS) (Taylor et al., “MicroRNA Signatures of Tumor-derived Exosomes as Diagnostic Biomarkers of Ovarian Cancer,” Gynecol Oncol 110(1): 13-21 (2008), which is hereby incorporated by reference in its entirety), nanomembrane ultrafiltration (Cheruvanky et al., “Rapid Isolation of Urinary Exosomal Biomarkers using a Nanomembrane Ultrafiltration Concentrator,” Am J Physiol Renal Physiol 292(5): F1657-61 (2007), which is hereby incorporated by reference in its entirety), immunoabsorbent capture, affinity purification, microfluidic separation, asymmetric flow field-flow fractionation (AF4) (Fraunhofer et al., “The Use of Asymmetrical Flow Field-Flow Fractionation in Pharmaceutics and Biopharmaceutics,” European Journal of Pharmaceutics and Biopharmaceutics 58:369-383 (2004); Yohannes et al., “Asymmetrical Flow Field-Flow Fractionation Technique for Separation and Characterization of Biopolymers and Bioparticles,” Journal of Chromatography. A 1218:4104-4116 (2011), which are hereby incorporated by reference in their entirety), or combinations thereof.

In one embodiment, the extracellular vesicles and particles are separated using a method that involves subjecting the sample to at least three sequential centrifugations. By way of example, and as described in the Examples infra, first, cell contamination may be removed from 3-4 day cell culture supernatant, bodily fluids or resected tissue culture supernatant by centrifugation at 500×mg for 10 minutes. Apoptotic bodies and large cell debris may then be removed by centrifuging the supernatants at 3,000×g for 20 minutes, followed by centrifugation at 12,000×g for 20 minutes to remove large microvesicles. Finally, exosomes are collected by spinning at 100,000×g for 70 minutes.

In one embodiment, the extracellular vesicles and particles are separated from a sample using a method that involves contacting the sample with one or more binding molecules capable of binding to alpha-2-macroglobulin, moesin, and galectin-3-binding protein. The complex of the binding molecule bound to extracellular vesicles and particles is separated from the sample. In one embodiment, the sample is contacted with one or more antibodies capable of binding to alpha-2-macroglobulin, moesin, and galectin-3-binding protein.

In another embodiment, the sample is contacted with a binding molecule capable of binding alpha-2-macroglobulin, a binding molecule capable of binding moesin, and a binding molecule capable of binding galectin-3-binding protein. In one embodiment, the binding molecules capable of binding alpha-2-macroglobulin, moesin, and galectin-3-binding protein are antibodies.

As used herein, a “binding molecule” may include an antibody or binding fragment thereof, or an antibody derivative that binds specifically to the protein of interest.

An antibody of the present disclosure is an intact immunoglobulin as well as a molecule having an epitope-binding fragment thereof that binds to a portion of the amino acid sequence of the protein of interest. As used herein, the terms “fragment”, “region”, and “domain” are generally intended to be synonymous, unless the context of their use indicates otherwise. Full antibodies typically comprise a tetramer which is usually composed of at least two heavy (H) chains and at least two light (L) chains. Each heavy chain is comprised of a heavy chain variable (VH) region and a heavy chain constant (CH) region, usually comprised of three domains (CH1, CH2 and CH3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable (VL) region and a light chain constant (CL) region.

Antibody fragments (including Fab and (Fab)2 fragments) that exhibit epitope-binding ability can be utilized and are obtained, for example, by protease cleavage of intact antibodies. Examples of the epitope-binding fragments suitable for use in the methods described herein include (i) Fab′ or Fab fragments, which are monovalent fragments containing the VL, VH, CL and CH1 domains; (ii) F(ab′)2 fragments, which are bivalent fragments comprising two Fab fragments linked by a disulfide bridge at the hinge region; (iii) Fd fragments consisting essentially of the VH and CH1 domains; (iv) Fv fragments consisting essentially of a VL and VH domain, (v) dAb fragments (Ward et al. “Binding Activities Of A Repertoire Of Single Immunoglobulin Variable Domains Secreted From Escherichia coli,” Nature 341:544-546 (1989) which is hereby incorporated by reference in its entirety), which consist essentially of a VH or VL domain and also called domain antibodies (Holt et al. “Domain Antibodies: Proteins For Therapy,” Trends Biotechnol. 21(11):484-490 (2003), which is hereby incorporated by reference in its entirety); (vi) camelid or nanobodies (Revets et al. “Nanobodies As Novel Agents For Cancer Therapy,” Expert Opin. Biol. Ther. 5(1):111-124 (2005), which is hereby incorporated by reference in its entirety) and (vii) isolated complementarity determining regions (CDR). An epitope-binding fragment may contain 1, 2, 3, 4, 5 or all 6 of the CDR domains of such antibody.

Antibody derivatives suitable for use in the methods disclosed herein include those molecules that contain at least one epitope-binding domain of an antibody, and are typically formed using recombinant techniques. One exemplary antibody derivative includes a single chain Fv (scFv). A scFv is formed from the two domains of the Fv fragment, the VL region and the VH region, which are encoded by separate gene.

Once plasma extracellular vesicles and plasma extracellular vesicle proteins are isolated from the biological sample using the methods described supra, the resulting plasma extracellular vesicle and plasma extracellular vesicle protein samples are subjected to a detection assay suitable for detecting the various protein biomarkers as described supra.

In accordance with all aspects of the disclosure relating to subjecting the plasma extracellular vesicle and plasma extracellular vesicle protein samples to a detection assay, suitable detection assays include, but are not limited to, those that measure protein expression levels. Methods for detecting and measuring protein expression levels generally involve an immunoassay, where the plasma extracellular vesicle protein sample is contacted with one or more detectable binding reagents that is suitable for measuring protein expression, e.g., a labeled antibody that binds to the protein of interest, i.e., a biomarker as described herein, or a primary antibody that binds to a biomarker used in conjunction with a secondary antibody. The one or more binding reagents bound to the biomarker (i.e., a binding reagent-biomarker complex) in the sample is detected, and the amount of labeled binding reagent that is detected and normalized to total protein in the sample, serves as an indicator of the amount or expression level of the biomarker present in the sample.

Suitable immunoassays for detecting protein expression level in an plasma extracellular vesicle sample that are commonly employed in the art include, for example and without limitation, western blot, immunoprecipitation, enzyme-linked immunosorbent assay (ELISA), radioimmunoassay (RIA), fluorescent activated cell sorting (FACS), immunoradiometric assay, gel diffusion precipitation reaction, immunodiffusion assay, in situ immunoassay, imaging mass cytometry, complement fixation assay, and immunoelectrophoresis assay.

In another embodiment, biomarker expression levels are measured using one-dimensional and two-dimensional electrophoretic gel analysis, high performance liquid chromatography (HPLC), reverse phase HPLC, Fast protein liquid chromatograph (FPLC), mass spectrometry (MS), tandem mass spectrometry, liquid crystal-MS (LC-MS), surface enhanced laser desorption/ionization (SELDI), MALDI, and/or protein sequencing.

In accordance with all aspects of the disclosure, protein biomarker expression levels, can also or alternatively be measured by detecting and quantifying biomarker nucleic acid levels using a nucleic acid detection assay. In one embodiment, RNA, e.g., mRNA, levels are measured. RNA is preferably reverse-transcribed to synthesize complementary DNA (cDNA), which is then amplified and detected or directly detected. The detected cDNA is measured and the levels of cDNA serve as an indicator of the RNA or mRNA levels present in a sample. Reverse transcription may be performed alone or in combination with an amplification step, e.g., reverse transcription polymerase chain reaction (RT-PCR), which may be further modified to be quantitative, e.g., quantitative RT-PCR as described in U.S. Pat. No. 5,639,606, which is hereby incorporated by reference in its entirety.

It may be beneficial or otherwise desirable to extract RNA from the primary tumor cells prior to or for analysis. RNA molecules can be isolated from cells and the concentration (i.e., total RNA) quantified using any number of procedures, which are well-known in the art, the particular extraction procedure chosen based on the particular biological sample. In some instances, with some techniques, it may also be possible to analyze the nucleic acid without extraction from the cells.

In one embodiment, mRNA is analyzed directly without an amplification step. Direct analysis may be performed with different methods including, but not limited to, nanostring technology (Geiss et al. “Direct Multiplexed Measurement of Gene Expression with Color-Coded Probe Pairs,” Nat Biotechnol 26(3): 317-25 (2008), which is hereby incorporated by reference in its entirety). Nanostring technology enables identification and quantification of individual target molecules in a biological sample by attaching a color coded fluorescent reporter to each target molecule. This approach is similar to the concept of measuring inventory by scanning barcodes. Reporters can be made with hundreds or even thousands of different codes allowing for highly multiplexed analysis. In another embodiment, direct analysis can be performed using immunohistochemical techniques.

In another embodiment, it may be beneficial or otherwise desirable to reverse transcribe and amplify the RNA prior to detection/analysis. Methods of nucleic acid amplification, including quantitative amplification, are commonly used and generally known in the art. Quantitative amplification will allow quantitative determination of relative amounts of RNA in the cells.

Nucleic acid amplification methods include, without limitation, polymerase chain reaction (PCR) (U.S. Pat. No. 5,219,727, which is hereby incorporated by reference in its entirety) and its variants such as in situ polymerase chain reaction (U.S. Pat. No. 5,538,871, which is hereby incorporated by reference in its entirety), quantitative polymerase chain reaction (U.S. Pat. No. 5,219,727, which is hereby incorporated by reference in its entirety), nested polymerase chain reaction (U.S. Pat. No. 5,556,773), self sustained sequence replication and its variants (Guatelli et al. “Isothermal, In vitro Amplification of Nucleic Acids by a Multienzyme Reaction Modeled after Retroviral Replication,” Proc Natl Acad Sci USA 87(5): 1874-8 (1990), which is hereby incorporated by reference in its entirety), transcriptional amplification and its variants (Kwoh et al. “Transcription-based Amplification System and Detection of Amplified Human Immunodeficiency Virus type 1 with a Bead-Based Sandwich Hybridization Format,” Proc Natl Acad Sci USA 86(4): 1173-7 (1989), which is hereby incorporated by reference in its entirety), Qb Replicase and its variants (Miele et al. “Autocatalytic Replication of a Recombinant RNA.” J Mol Biol 171(3): 281-95 (1983), which is hereby incorporated by reference in its entirety), cold-PCR (Li et al. “Replacing PCR with COLD-PCR Enriches Variant DNA Sequences and Redefines the Sensitivity of Genetic Testing.” Nat Med 14(5): 579-84 (2008), which is hereby incorporated by reference in its entirety) or any other nucleic acid amplification method known in the art. Depending on the amplification technique that is employed, the amplified molecules are detected during amplification (e.g., real-time PCR) or subsequent to amplification using detection techniques known to those of skill in the art. Suitable nucleic acid detection assays include, for example and without limitation, northern blot, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, and mass spectrometry (MS) methods (e.g., MassARRAY® System).

In various related aspects, the present disclosure also relates to kits for performing the methods described herein. Such kits contain reagents and procedures that can be utilized in a clinical or research setting or adapted for either the field laboratory or on-site use. In particular, kits comprising the disclosed reagents used in practicing the methods described herein include any of a number of means for detecting the proteins of interest and measuring the presence or absence of such proteins, along with appropriate instructions, are contemplated. Suitable kits comprise reagents sufficient for performing an assay to detect a protein of interest including, without limitation, antibodies and fragments thereof.

It is to be understood that such a kit is useful for any of the methods of the present disclosure. The choice of particular components is dependent upon the particular method the kit is designed to carry out. Additional components can be provided for detection of the analytical output.

As described above, the kit optionally further comprises instructions for detecting the proteins of interest by the methods described herein. The instructions present in such a kit instruct the user on how to use the components of the kit to perform the various methods of the present disclosure. These instructions can include a description of the detection methods of the present disclosure.

Another aspect of the present disclosure is directed to a kit suitable for detecting, in a liquid biopsy sample from a subject, the presence of cancer in subject. The kit includes reagents, e.g., detectable binding molecules, suitable for detecting: (i) plasma extracellular vesicle and/or (ii) a plasma extracellular vesicle protein selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

In accordance with all aspects of the disclosure, the cancer is selected from the group consisting of lung cancer, prostate cancer, breast cancer, liver cancer, pancreatic cancer, kidney cancer, colon cancer, ovarian cancer, skin cancer, or a sarcoma. In some embodiments, the cancer is lung cancer. In some embodiments, the cancer is prostate cancer. In some embodiments, the cancer is breast cancer. In some embodiments, the cancer is liver cancer. In some embodiments, the cancer is pancreatic cancer. In some embodiments, the cancer is kidney cancer. In some embodiments, the cancer is colon cancer. In some embodiments, the cancer is ovarian cancer. In some embodiments, the cancer is skin cancer. In some embodiments, the cancer is a sarcoma.

In some embodiments, the sarcoma is selected from the group consisting of angiosarcoma, epitheloid sarcoma, leiomyosarcoma, liposarcoma, myxofibrosarcoma, osteosarcoma, and undifferentiated pleomorphic sarcoma/malignant fibrous histiocytoma.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Overview of Study Cohort Populations

Plasma was acquired from a cohort of 40 sarcoma patients on the doxorubicin monotherapy control arm of the international, open-label, prospective, randomized, phase 3, multicenter SARC021 trial (TH CR-406)¹⁶. SARC021 was conducted at 81 academic or community investigational sites across 13 countries, and one arm of the trial treated patients with doxorubicin alone, the standard of care. Samples were obtained from 8 different sarcoma subtypes in the cohort, including angiosarcoma (AS), epitheloid sarcoma (EPI), leiomyosarcoma (LMS), liposarcoma (LS), myxofibrosarcoma (MFS), malignant peripheral nerve sheath tumor (MPNST), rhabdomyosarcoma (PMRMS), and undifferentiated pleomorphic sarcoma (UPS). The number of patients per sarcoma subtype ranged from 1 to 13 (Table 1).

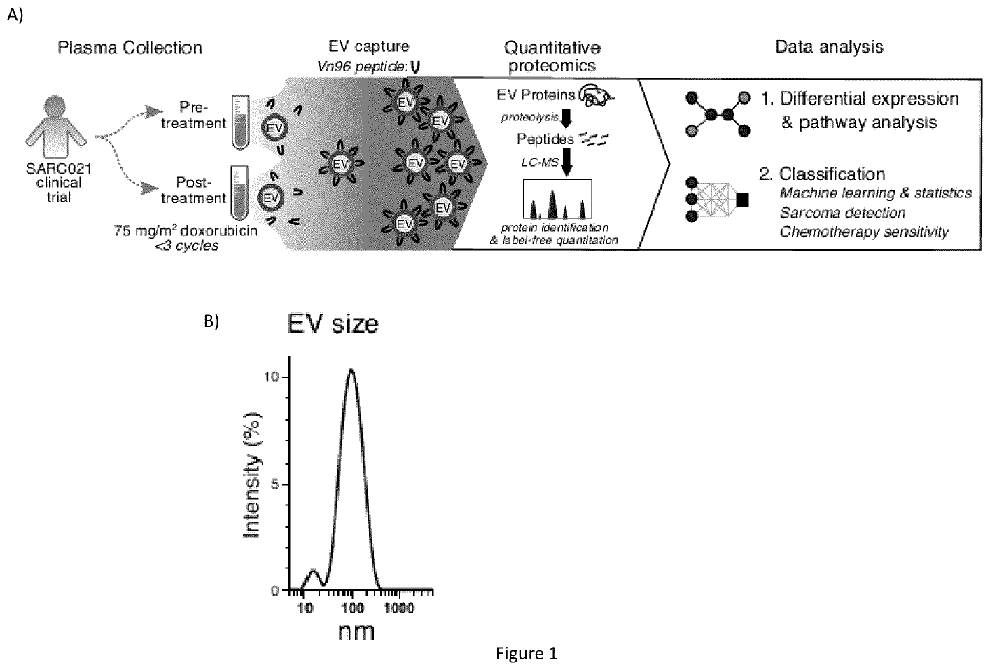

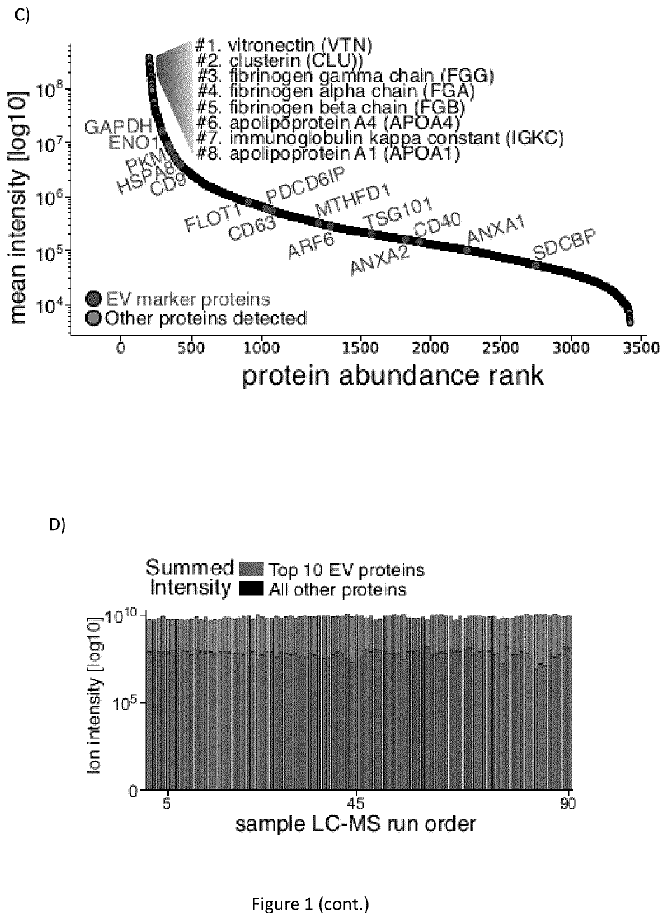

Plasma for the sarcoma cohort was acquired prior to treatment with two cycles of 75 mg/m² doxorubicin, and just prior to cycle 3, for a total treatment time of 6 weeks (FIG. 1A). 15 of the sarcoma patients (37.5%) were considered treatment resistant based on RECIST version 1.1 guidelines^{16,18} (Table 1). There were no significant differences in the gender, age, or race of the patients that were sensitive versus resistant to treatment.

TABLE 1
Patient and clinic characteristics summary of sarcoma patients.

| | | Overall | Resistant | Sensitive | P- |
|---|---|---|---|---|---|
| Variable | Level | N = 40 | N = 15 | N = 25 | Value* |
| Sex | F | 15 | 4 | 11 | 0.273 |
| | | (37.50%) | (26.67%) | (44.00%) | |
| | M | 25 | 11 | 14 | |
| | | (62.50%) | (73.33%) | (56.00%) | |
| Histology | AS | 1 | 0 | 1 | 0.805 |
| | | (2.50%) | (0.00%) | (4.00%) | |
| | EPI | 2 | 1 | 1 | |
| | | (5.00%) | (6.67%) | (4.00%) | |
| | LMS | 6 | 2 | 4 | |
| | | (15.00%) | (13.33%) | (16.00%) | |
| | LS | 11 | 4 | 7 | |
| | | (27.50%) | (26.67%) | (28.00%) | |
| | MFS | 1 | 0 | 1 | |
| | | (2.50%) | (0.00%) | (4.00%) | |
| | MPNST | 4 | 3 | 1 | |
| | | (10.00%) | (20.00%) | (4.00%) | |
| | PMRMS | 2 | 0 | 2 | |
| | | (5.00%) | (0.00%) | (8.00%) | |
| | UPS/MFH | 13 | 5 | 8 | |
| | | (32.50%) | (33.33%) | (32.00%) | |
| Age | 30-39 | 5 | 3 | 2 | 0.607 |
| | | (12.50%) | (20.00%) | (8.00%) | |
| | 40-49 | 4 | 2 | 2 | |
| | | (10.00%) | (13.33%) | (8.00%) | |
| | 50-59 | 9 | 4 | 5 | |
| | | (22.50%) | (26.67%) | (20.00%) | |
| | 60-69 | 9 | 3 | 6 | |
| | | (22.50%) | (20.00%) | (24.00%) | |
| | 70-79 | 13 | 3 | 10 | |
| | | (32.50%) | (20.00%) | (40.00%) | |
| Race | AMERICAN INDIAN | 1 | 0 | 1 | 0.767 |
| | OR ALASKA NATIVE | (2.50%) | (0.00%) | (4.00%) | |
| | ASIAN | 1 | 1 | 0 | |
| | | (2.50%) | (6.67%) | (0.00%) | |
| | BLACK OR AFRICAN | 1 | 0 | 1 | |
| | AMERICAN | (2.50%) | (0.00%) | (4.00%) | |
| | WHITE | 37 | 14 | 23 | |
| | | (92.50%) | (93.33%) | (92.00%) | |

Patient/clinic characteristics summary of the 40 sarcoma patients. There were no significant sex, histology, age and race difference between sensitive and resistant sarcoma tumors.
*p-values: Fisher's exact test or Chi-square test as proper compared characteristics between resistant and sensitive sarcoma tumors.

Plasma EVs from 10 healthy volunteers were acquired from non-oncology clinical trials as controls. All studies were approved by local institutional review boards. Additionally, a plasma EV proteomic dataset from cancer patients published by Hoshino et al. served as an independent validation cohort with an additional 82 cancer patients and 45 healthy volunteers¹. While the Hoshino dataset included 5 osteosarcoma patients, it primarily consists of carcinomas including breast, colorectal, ependymoma, germinoma, lung, lymphoma, melanoma, neuroblastoma, pancreatic, and stomach, as well as Wilms tumor, as detailed in reference¹. Notably, the Hoshino cohort was assayed by independent laboratories, employed a different EV enrichment method, and utilized different LC-MS instrumentation and proteomic data analysis methods than our sarcoma cohort.

### Example 2—Characterization of the Sarcoma Plasma EV Proteome

The plasma EV proteome of the Sarcoma cohort was quantitatively profiled using a robust and efficient EV enrichment and label free proteomics workflow previously employed to characterize plasma EVs in mouse models¹⁷ (FIG. 1A). This study design enables a ‘rectangular’ approach to discover multiparametric biomarkers, assaying as many proteins as possible in each sample, an alternative to more standard ‘triangular’ workflows that focus on discovering a single biomarker in a small number of samples followed by immunoassays in validation cohorts¹⁹. There are many advantages to a ‘rectangular’ biomarker discovery strategy, including amenability to machine learning and other computational approaches that can unbiasedly reveal diagnostic proteomic signatures^{8,20}.

Plasma EVs were isolated using the well-characterized Vn96 EV capture technology²¹⁻²³. Advantages of the VN96 approach include its simplicity, robustness, and limited contamination from plasma lipoproteins compared to alternative purification methods^{17,24,25}. The overall size of isolated plasma EVs was determined by dynamic light scattering (DLS) to fully sample the broad range of vesicle sizes (1-10,000 nm diameter) that may be present in complex specimens such as plasma. Most EVs were between 40-275 nm in diameter with a peak near 100 nm (FIG. 1B). This size distribution is consistent with robust enrichment of all EV subpopulations including exomeres, exosomes, and some microvesicles, but not large apoptotic bodies or intact cells²⁶.

Proteomic profiling of the enriched plasma EVs assigned 30,298 peptides to 3,477 protein families with a 1% FDR (Data not shown). On average, 1391 proteins were detected per sample with quantified protein intensities spanning 5 orders of magnitude (FIG. 1C). Most conventional EV marker proteins were detected, including CD9, CD63, PDCD6IP (ALIX), FLOT1, and TSG101. Consistent with EV size analysis, the exomere marker MTHFD1 was detected as well as markers of EV subpopulations such as ANXA1, a specific indicator of EVs shed from the plasma membrane^{26,27}. The most abundant proteins detected, VTN, CLU, and fibrinogens, are all routinely detected in EV sample preparations from plasma or cancer-related samples²⁸⁻³³. Label-free quantitation was consistent across the sample set, as indicated by the similar signal intensity of the top 10 EV proteins in the Exocarta database as well as the remainder of the peptides detected (FIG. 1D)^{28,29}.

### Example 3—The Proteome of Plasma EVS from Sarcoma Patients Indicates the Presence of Cancer

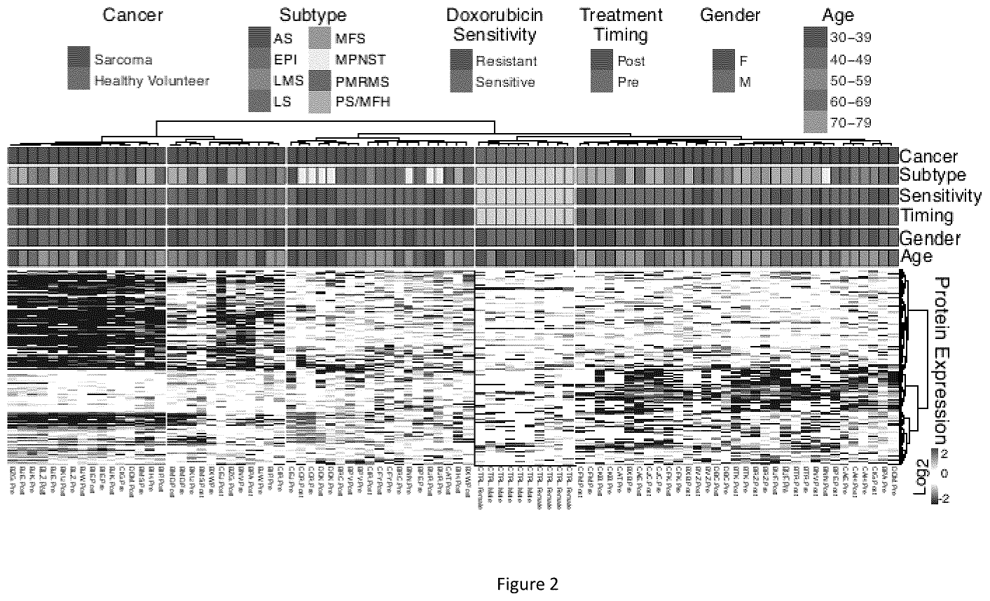

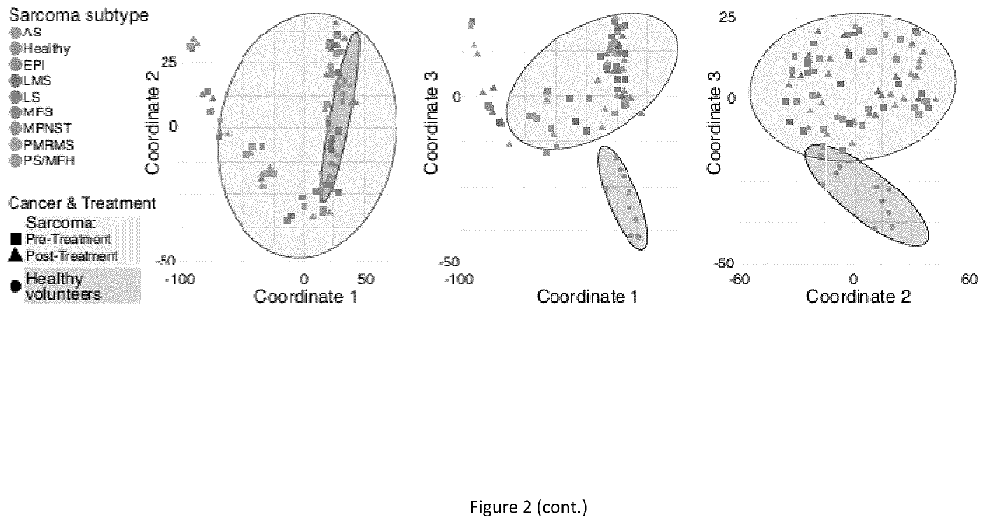

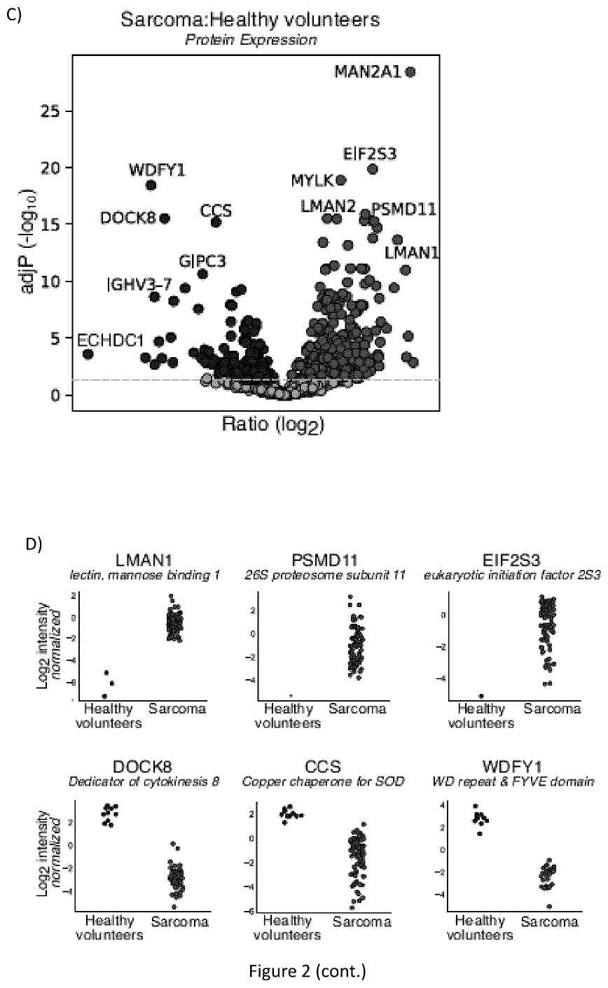

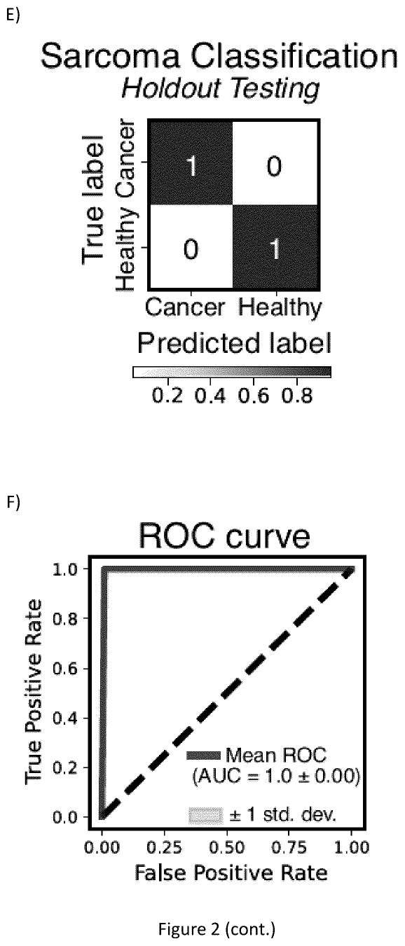

To explore global patterns in plasma EV protein expression of the sarcoma cohort, unsupervised hierarchical clustering of all 90 samples was conducted. All healthy volunteer samples clustered together, flanked on either side by ˜50% of the sarcoma patient samples (FIG. 2A). While the sarcoma plasma exhibited 3-4 distinct clusters, there was no clear hierarchical grouping of samples based on sarcoma subtype, drug sensitivity, timing (pre-versus post-treatment), gender, or age.

Unsupervised multidimensional scaling (MDS) is an orthogonal approach to unbiasedly classify sarcoma patients versus healthy volunteers. While the first two MDS coordinates alone did not distinguish sarcoma patients from heathy volunteers, MDS coordinate 3 enabled clear delineation of healthy volunteers and sarcoma patients in combination with either coordinates 1 or 2 (FIG. 2B). Clear clustering by sarcoma subtype, treatment timing (pre- vs. post-treatment), doxorubicin sensitivity, or any other factor was not observed, however.

To determine which protein markers best differentiated sarcoma patients from healthy volunteers, differential expression analysis of plasma EV proteins was performed by comparing healthy volunteers to sarcoma patients prior to doxorubicin treatment. After filtering, 399 proteins had significantly higher abundance in sarcoma patients, and 122 had significantly decreased abundance after multi-hypothesis correction of p-values (FIG. 2C). The expression of 44% of the proteins robustly detected was significantly altered in sarcoma patients, indicating that the presence of sarcoma induced widespread changes in the plasma EV proteome. Many of these differentially expressed proteins were robustly detected and have a wide range of known functions, including the lectin LMAN1, the proteasomal subunit PSMD11, and the eukaryotic initiation factor EIF2S3 which were all upregulated in sarcoma patients (FIG. 2D). Proteins such as DOCK8, CCS, and WDFY1 were also robustly detected in sarcoma patients at a lower abundance than in healthy volunteers. The plasma EV proteome coupled with the K-Nearest Neighbors (KNN) algorithm³⁴ classified samples from sarcoma patients prior to treatment from healthy volunteers with perfect accuracy and separation (AUROC) using ‘holdout’ testing methodology³⁵ across 7 randomized binning of the samples into training, validation, and testing sets as described in the Methods, indicating that coupling the plasma EV proteomics with machine learning has high diagnostic potential (FIG. 2E, F).

### Example 4—A Pan-Cancer Plasma EV Proteomic Atlas and Diagnostic Classifier

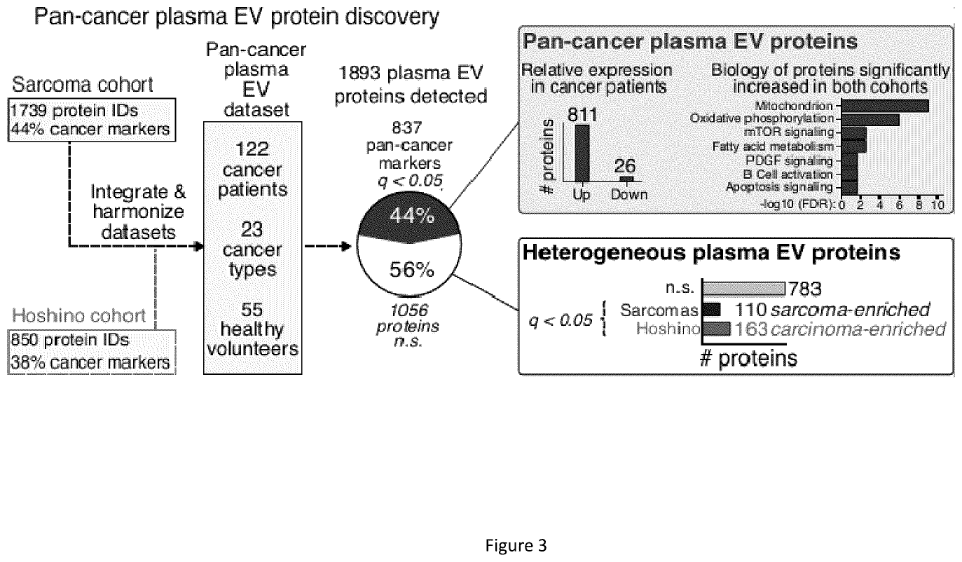

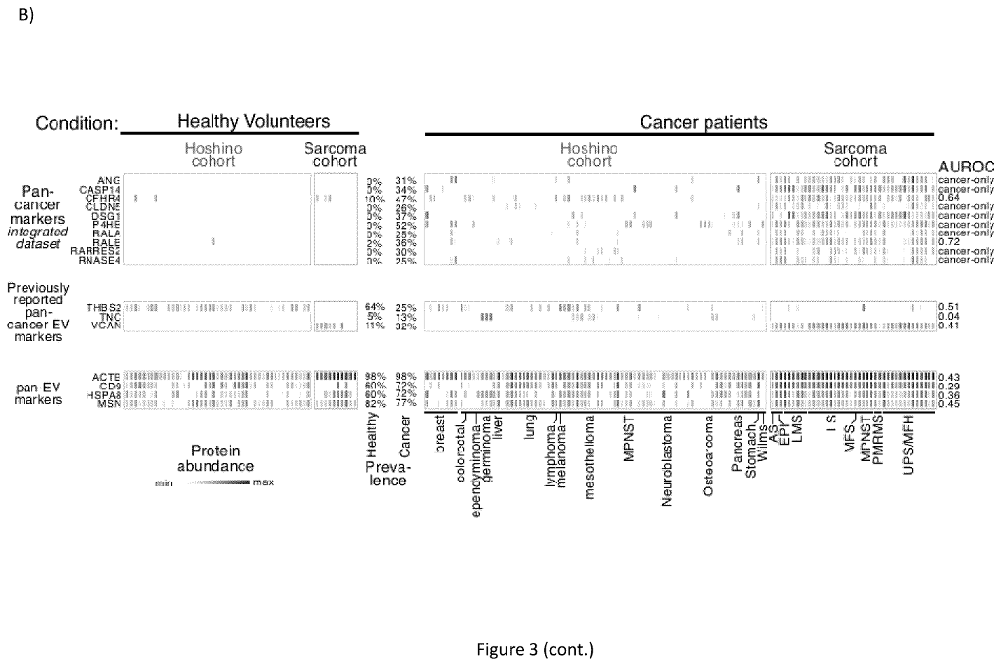

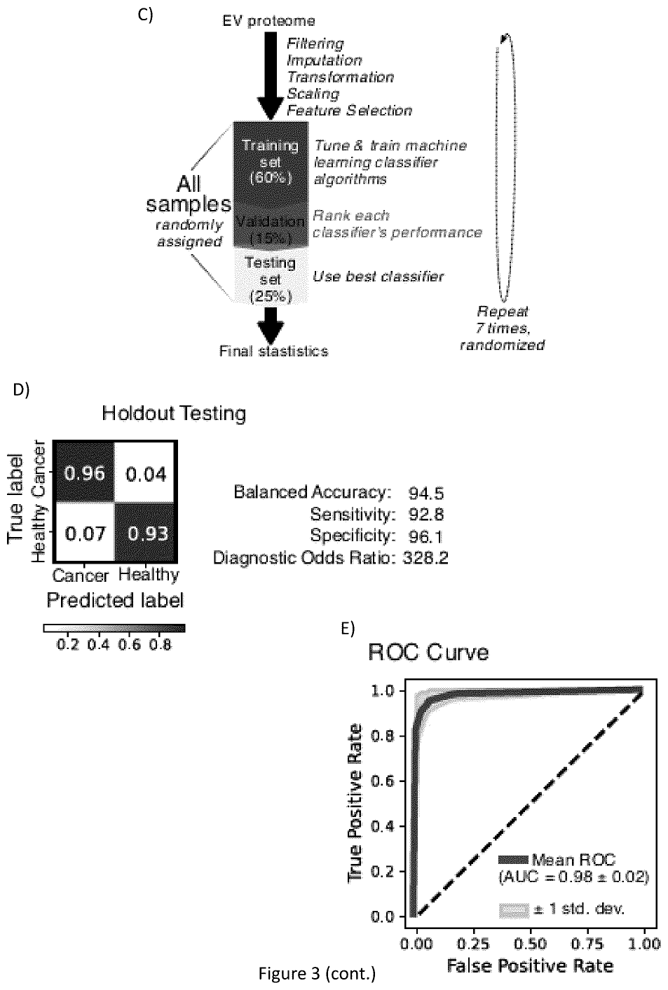

Sarcomas are a heterogenous group of malignant tumors that arise from mesenchymal cells, and the ˜175 pathological sarcoma subtypes exhibit remarkable differences in clinical behavior, response to treatment, genetics, and molecular profiles due to their diverse cell of origin³⁶. Since sarcomas can arise from muscle, bone, fat, blood vessels, nerves, and more, each sarcoma subtype is often as unique as a distinct cancer type^{37,38}. The absence of clear clustering by sarcoma subtype suggested that the sarcoma-dependent EV proteomic changes might be a hallmark shared across many cancer types. To investigate this, the results from the sarcoma cohort were compared to an independent plasma EV proteomic dataset reported by Hoshino et al. that primarily included carcinomas¹. We reasoned that plasma EV proteins with cancer-associated changes across this combined dataset of 122 cancer patients, 23 cancer types, and 55 healthy volunteers would represent the most comprehensive pan-cancer plasma EV proteomic signature to date and would be robust given the differences in EV enrichment methods, plasma collection, patient cohorts, and proteomic workflows between the datasets (FIG. 3A). Only samples from patients without treatment annotation were included from each dataset to limit differences due to treatment. One attribute shared by both datasets was that CD9 and HSPA8 were the most readily detected conventional EV markers, even though EVs were captured in the Hoshino cohort using ultracentrifugation and the Vn96 capture approach was applied for the sarcoma cohort (FIG. 1C).

Proteomic data from both cohorts were integrated with the HarmonizR software package to minimize batch effects and variance³⁹. After harmonization and filtering for detection in at least 15 samples, 1739 proteins were retained from the Sarcoma cohort, and 850 were retained from the Hoshino cohort, for a total of 1893 plasma EV proteins in the integrated, pan-cancer dataset. Widespread changes in the EV proteome in cancer patients were observed in both cohorts, with more than one third of the plasma proteome significantly changed in each individual dataset (FIG. 3A, q<0.05 Benjamini-Hochberg adjusted). Furthermore, the proteins altered in both cohorts were consistent and encompassed 837 pan-cancer markers, 44% of the plasma EV proteins detected (FIG. 3A, q<0.05 Benjamini-Hochberg adjusted). Of these proteins, the vast majority (811) were increased in cancer patient plasma, including five proteins (TGFB1, CAPN2, C1QBP, S100A11, CALML5) known to be released by tumors into plasma EVs in vivo¹⁷ (FIG. 3A). The magnitude and shared direction of these cancer-associated changes demonstrate that cancer has a far-reaching impact on plasma EVs in vivo. Intriguingly, more than 50 of the pan-cancer-regulated proteins are annotated to be exclusively localized extracellularly⁴⁰, indicating they are unlikely to be packaged within the EVs themselves and more likely to be localized to the EV surface. There were comparably few cohort-specific changes in the plasma EV proteome (273 proteins), which could result from the unique tumor types present in each cohort, the specific proteins detected, the EV enrichment method, or cohort-specific proteomic data acquisition and peptide-level preprocessing.

To characterize the biology of the pan-cancer EV proteome, over-representation analysis was performed using the expression of all 1893 proteins detected as the reference list. Analysis of the 811 significantly upregulated proteins revealed strong enrichment of biological annotations associated with mitochondria and oxidative phosphorylation, as well as apoptosis signaling (FIG. 3A). These are consistent with an upregulation of a recently defined EV subtype termed mitovesicles or mitoEVs that originate from mitochondria and are released by cancer cells to adapt to resource demands and restore growth^{41,42}. The other enriched biology functions, mTOR, PDGF, and B Cell signaling, all of which are hallmarks of cancer⁴³.

The widespread upregulation of plasma EV proteins by cancer suggested that pan-cancer plasma EV protein biomarkers may exist. A previous study focused on THBS2, TNC, and VCAN as putative pan-cancer markers¹, but these were relatively poor markers in the integrated atlas with AUROCs below 0.51, suggesting they were laboratory-or method-specific (FIG. 3B). However, using this interlaboratory datasets revealed several robust pan-cancer markers, including the beta-subunit of prolyl 4-hydroxylase (P4HB) which was detected in 52% of cancer samples across both datasets, but was undetected in plasma EVs from all healthy volunteers (FIG. 3B). Several other perfect classifiers of cancer detected in at least 25% of all cancer samples and in both datasets, include ANG, CASP14, CLDN5, DSG1, RALA, RRES2, and RNASE4 (FIG. 3B). CFHR4 and RALB were also highly enriched in plasma EVs from cancer patients with excellent classification performance individually.

The presence of pan-cancer EV protein markers suggested that a proteomic signature in plasma EVs could provide a pan-cancer liquid biopsy diagnostic assay. To generate a multivariate classifier, machine learning was coupled with a data analysis approach that split the entire dataset into 7 independent training, validation, and testing sets for a ‘heldout’ testing methodology³⁵ (FIG. 3C). As detailed in the Methods, the training set utilized cross validation to tune and train 17 separate machine learning algorithms such as support vector machines, gradient boosting algorithms, and a neural network. Each optimally tuned classifier was then evaluated on the heldout, independent Validation set primarily to identify overtrained classifiers. The best classifier was then used to evaluate the heldout Test set. Due to the stochastic nature of machine learning and sample binning, 7 analysis iterations were performed with random starting seeds and random assignment of samples to each group. However, summary data from the 7 Test sets is from a single classifier, the top ranked from the training and validation sets.

The predictive power of the plasma EV proteome to classify cancer patients from healthy volunteers was excellent. The K-nearest neighbors algorithm³⁴ classified cancer patients with all cancer types from healthy volunteers with a mean ROC AUC of 0.98, specificity of 96%, diagnostic odds ratio of 328, and balanced accuracy of 95% on the 7 heldout Test sets (FIG. 3D, E). This classification performance is slightly higher than previously reported using plasma EV proteins¹, likely due to using a larger protein atlas and recent advances in machine learning algorithms. It is also better than other pan-cancer-focused biomarker studies and on par with several that employ a targeted approach^{10,44-46}. The plasma EV proteome has clear diagnostic potential for cancer detection in patients, which could prove extremely useful for cancers that are difficult to detect early such as lung and pancreatic cancer. As a Resource, the KNN classifier trained on the entire integrated dataset is provided to classify any plasma EV proteomics dataset and has a cross-validation accuracy and AUROC of 97.2% and 0.98 AUROC, respectively, for cancer detection.

The Following Example 5—The Plasma EV Proteome Defines Multiple Cancer Types

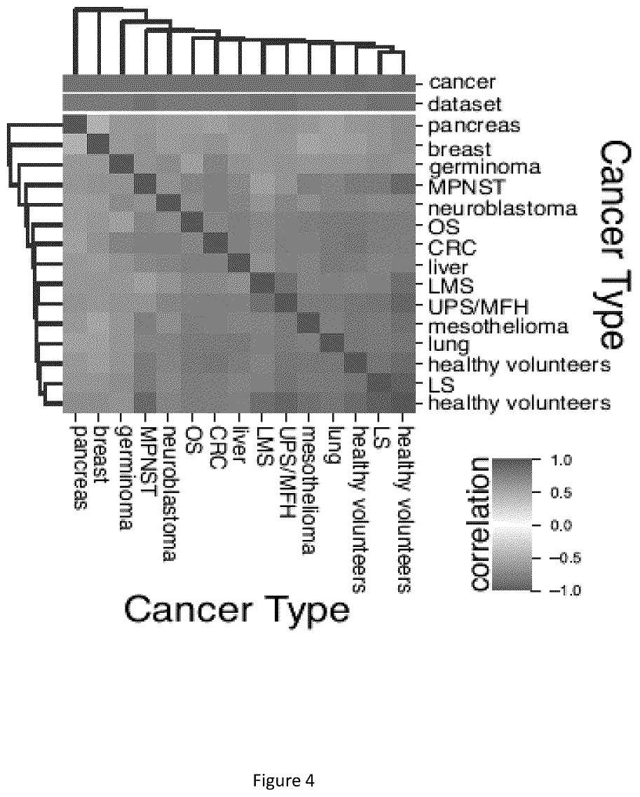

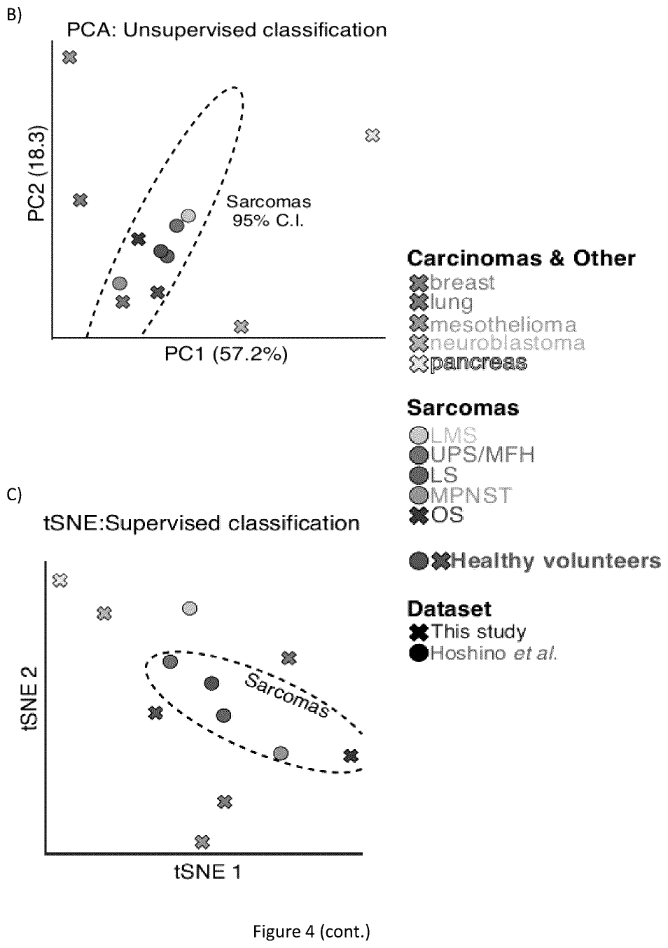

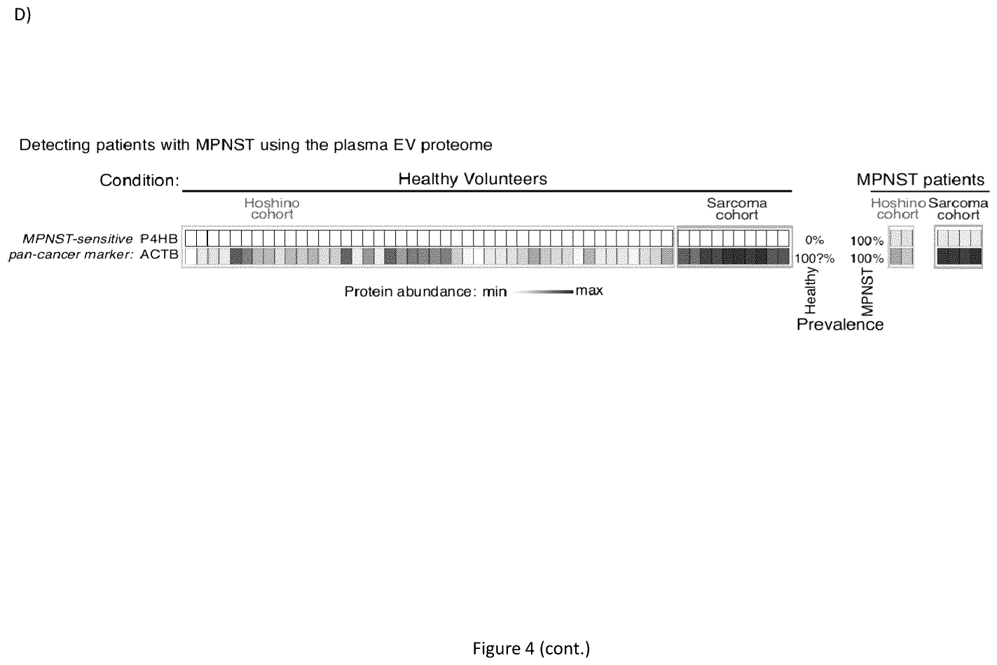

Tumor-type-demarcating plasma EV proteins have the potential to improve cancer diagnosis, especially cases of unknown primary origin⁴⁷. The low correlation of plasma EV expression across cancer types suggested that unique proteomic markers defining cancer types may be present (FIG. 4A). While robust clustering by subtype was not observed in the sarcoma cohort, Hoshino et al. identified tumor-specific signatures in the plasma EV proteome of their cohort¹ and other studies have shown potential to distinguish different cancer types based on plasma markers^{10,44,46,48}. Therefore, principal component analysis (PCA) was performed to unbiasedly classify each cancer subtype with at least 4 patient samples in the integrated dataset. Pancreatic cancer, breast cancer, mesothelioma, and neuroblastoma were clearly differentiated from other cancer types, with 75.5% of the variance explained by the first 2 principal components (FIG. 4B). Healthy volunteers from both datasets were tightly clustered, indicating effective data harmonization and consistent plasma EV proteome abundance across EV sample preparation methods and analytical methods. Since sarcomas from both cohorts were clustered near the healthy volunteers and lung cancers, PCA was complemented with a supervised clustering approach using t-distributed stochastic neighbor embedding (t-SNE). T-SNE fully separated lung cancers from other cancer types, and separated sarcomas from healthy volunteers better, further underscoring the utility of the plasma EV proteome for cancer categorization (FIG. 4C).

MPNST is a rare, highly aggressive sarcoma that would benefit from a non-invasive biomarker since biopsies can lead to peripheral nerve injury. Since MPNSTs were found in both the sarcoma and Hoshino cohorts, we investigated if a novel, non-invasive biomarker for this rare cancer was present in the plasma EV proteome. When no MPNST-unique marker was present, the pan-cancer marker P4HB was detected in all MPNST samples across both cohorts, but none from healthy volunteers, suggesting this is an especially promising marker to detect MPNST (FIG. 4D).

### The Following Example 6—The Plasma EV Proteome Predicts Sarcoma Response to Doxorubicin Prior to Treatment

The plasma EV proteome has potential to non-invasively predict drug treatment responses, especially responses to potentially toxic chemotherapies such as doxorubicin. However, the plasma EV proteome has not been harnessed to date as a secondary objective correlate in prospective clinical trials where several timepoints are acquired despite evidence that chemotherapy-induced extracellular vesicles (chemo-EVs) have been proposed to carry different cargo loads than non-chemotherapy-induced EVs⁴⁹ and exosomes from doxorubicin-resistant MCF-7 breast cancer cells can transmit chemoresistance via horizontal transfer of microRNAs⁵⁰. While long RNA profiles in plasma EVs have been used to construct high accuracy diagnostic signatures of doxorubicin treatment⁵¹ and plasma EV microRNAs can predict doxorubicin-induced cardiotoxicity in dogs⁵², proteomic analysis of EVs in the setting of doxorubicin treatment in sarcoma has not been investigated to date.

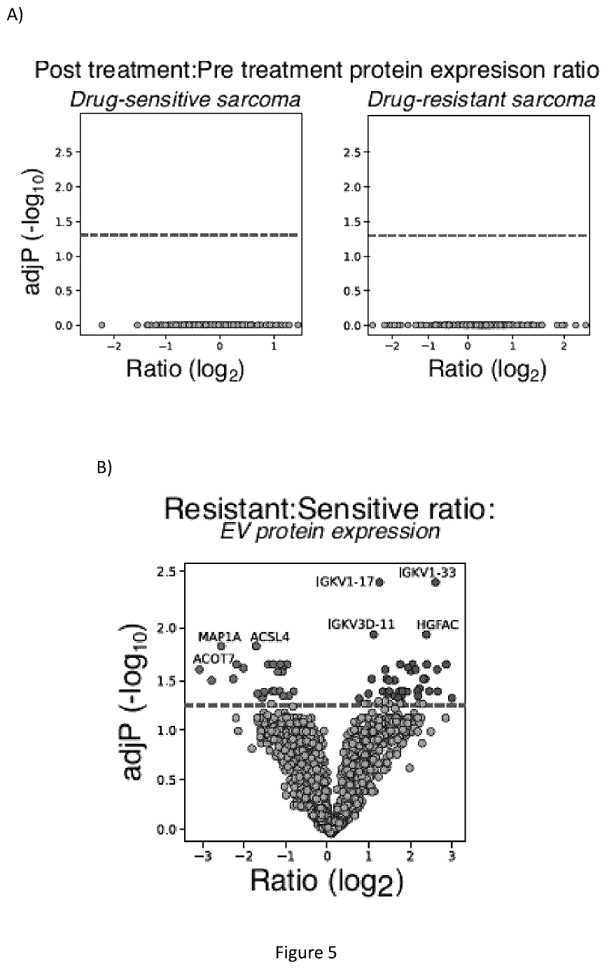

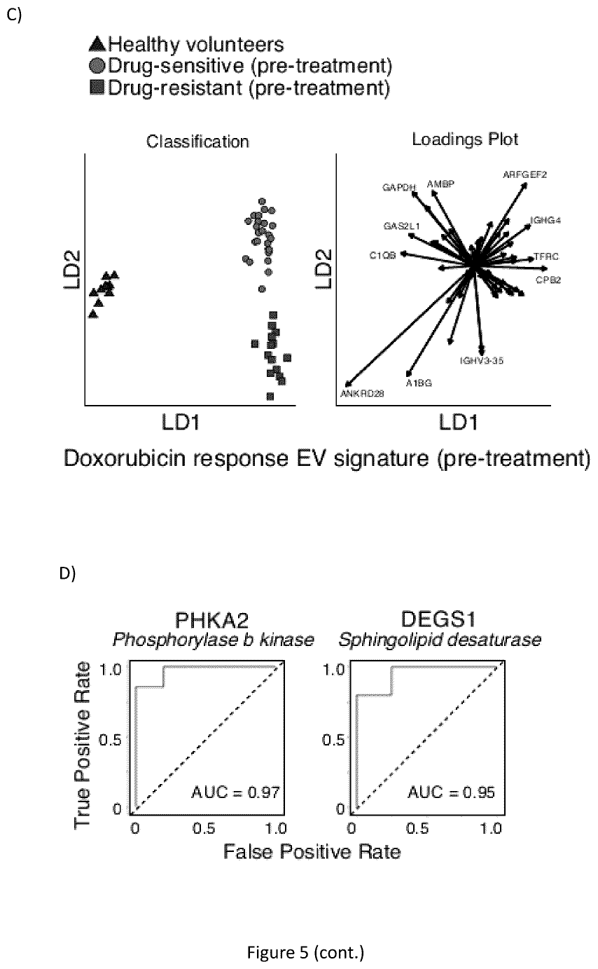

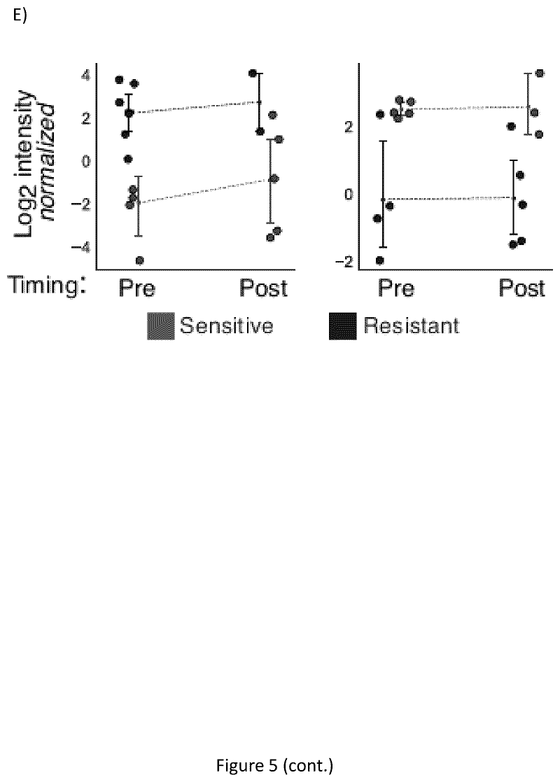

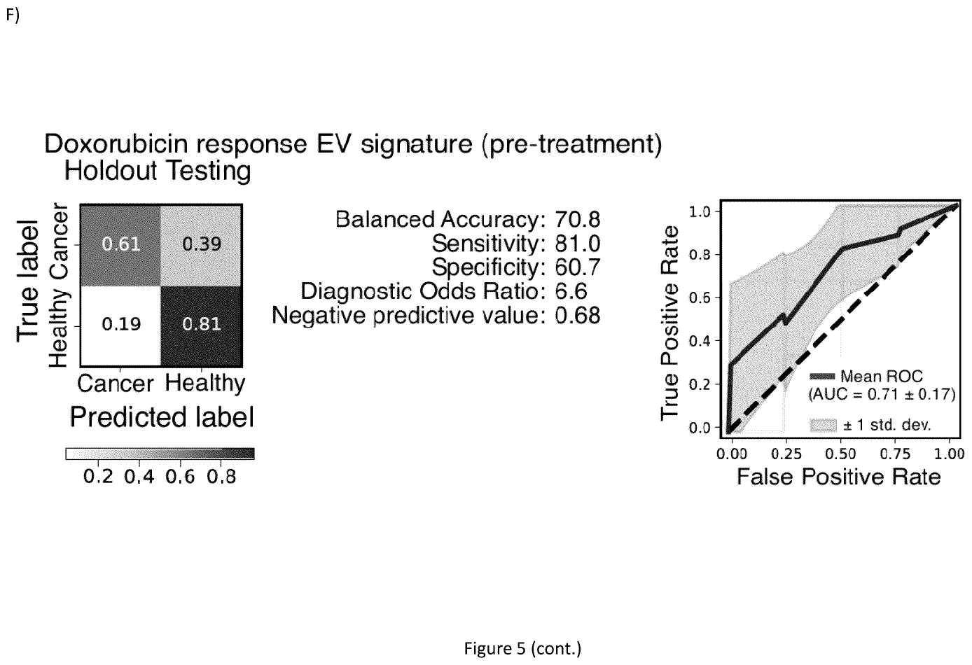

Doxorubicin is a topoisomerase isomerase inhibitor that perturbs all dividing cells in the body and has broad potential to alter the EV proteome in patients. However, no statistically significant change in EV protein abundance was observed when comparing plasma samples collected before and after treatment in the SARC021 sarcoma cohort, even considering the patient's treatment sensitivity (FIG. 5A). Despite the absence of a treatment effect, 38 proteins had significantly higher abundance in doxorubicin-resistant patients, most prominently the immunoglobulins IGKV1-17, IGKV1-33, and IGV3D-11 (FIG. 5B, q<0.05 Benjamini-Hochberg adjusted). 25 proteins had significantly lower expression in doxorubicin-resistant patients, including MAP1A, ACSL4, and ACOT7.

The absence of an adaptive response to doxorubicin treatment suggested that an intrinsic set of plasma EV marker proteins present prior to treatment may indicate treatment response. Indeed, supervised linear discriminant analysis (LDA) of the plasma EV proteome prior to treatment perfectly classified both the presence of sarcoma and treatment response (FIG. 5C). The loadings plot indicates that the drivers of classification include a mix of secreted, extracellular proteins (IGHV3-55, IGHG4, C1QB, CPB2) as well as other proteins that were intracellular (ANKRD28, GAPDH, GAS2L1). To evaluate the diagnostic potential of univariate plasma EV protein biomarkers to predict doxorubicin sensitivity in sarcoma patients, ROC analysis of all proteins was performed. (Data not shown). 278 proteins had an AUROC greater than 0.90, and 655 proteins significantly differentiated doxorubicin-resistant from-sensitive patients, indicating that the plasma EV proteome has high capability of distinguishing which patients would respond to therapy. For example, PHKA2 and DEGS1 are excellent classifiers of doxorubicin treatment sensitivity and are intrinsic markers of sensitivity, but their expression is not affected by treatment (FIG. 5D, E).

To generate a prognostic EV proteomic classifier with predictive power for future datasets, a multivariate plasma EV proteomic signature of doxorubicin treatment sensitivity prior to treatment was generated by machine learning using the approach shown in FIG. 3C. The XGBoost algorithm had the best overall performance on the training and validation datasets, and was used for all 7 randomized iterations of the pipeline⁵³. Diagnostic performance characteristics on the heldout Test set include an average balanced accuracy of 71%, sensitivity of 81%, specificity of 61%, diagnostic odds ratio of 6.6, and a negative predictive value of 0.68 (FIG. 5F). This predictive classification performance is on par with the best predictor of doxorubicin sensitivity in breast cancer patients which uses PET/MRI⁵⁴. While this predictive power pre-treatment is modest, its has high clinical significance since it is noninvasive and can spare patients from suboptimal treatment outcomes and unnecessary cardiotoxicity⁵⁵ to the standard of care⁵⁶.

### The Following Example 7—An Atlas of Plasma Extracellular Vesicle Proteins Reveals Markers of Pan-Cancer Identification and Doxorubicin Sensitivity

A comprehensive atlas of pan-cancer plasma EV proteins was established that enabled development of both a multiparameter pan-cancer classifier and a predictive model for doxorubicin response in sarcoma patients prior to treatment. Prospective clinical evaluation of this workflow combining streamlined plasma EV purification, robust proteomic sample preparation and acquisition, and advanced data processing could potentially eliminate doxorubicin toxicity by identifying intrinsically resistant patients before treatment initiation. This approach is readily scalable to large patient cohorts as it works effectively with frozen plasma samples and can profile approximately 1,850 proteins from human plasma EVs even in complex international, multi-center clinical trials like SARC-021.

Pan-cancer diagnostic studies have predominantly focused on circulating cell-free DNA (cfDNA), cell-free RNA (cfRNA), and circulating tumor cells (CTCs)¹⁰. However, these biomarkers generally exhibit limited sensitivity, partly due to their plasma instability and restricted tumor release^{10,57-59}. Protein panels from plasma^{48,58} and plasma EVs1 can detect multiple cancer types, often demonstrating superior sensitivity and classification performance compared to cfDNA, cfRNA, and CTCs¹⁰. Given that EV-associated proteins serve critical functional roles in cancer growth, metastasis, and drug resistance²⁻⁵, the multi-laboratory pan-cancer proteomic atlas we generated offers significant value for both mechanistic and translational cancer research^{2,60}. A limitation worth noting is the retrospective nature of this analysis; EV proteomic profiling must be incorporated into prospective studies to better assess its predictive capabilities. Additionally, like most global proteomics studies, our analysis relies on relative quantitation and implementing absolute protein quantitation could enhance rigor and robustness. Combining EV proteomics with complementary assays of CTCs or cfDNA, which offer very high specificity, may further reduce false discoveries.

The present findings that immunoglobulins and extracellular proteins are significantly altered by cancer and indicate treatment resistance support the emerging view that proteins adsorbed to EV surfaces, not just the internal EV cargo, play crucial roles in EV biology and diagnostics^{1,61,62}. These adsorbed proteins likely originate from non-tumor tissues, as studies show tumors contribute minimally (<1%) to the plasma EV proteome in mouse xenograft models¹⁷. This limited tumor contribution to plasma EVs seems at odds with the 40-fold increase in plasma EVs in breast cancer patients^{8,63}, the broad detectability of cancer-related EV biomarkers^{1,8,10}, and the extensive changes in plasma EV proteomes observed I this study and elsewhere^{1,8}. The present results help reconcile this discrepancy, as at least 50 pan-cancer-regulated proteins are exclusively extracellular and may form the protein corona on EV surfaces rather than existing as internal cargo. Cancer may also regulate distant tissues to prompt these non-tumor cells to produce “cancer-specific” EVs that contribute significantly to biological processes and biomarker profiles despite not originating from tumors themselves. EVs appear to function as signaling hubs with dynamic, regulated, and diagnostically relevant protein interactions on their exterior surfaces, where cancer-specific adapters may passively adsorb or actively bind circulating plasma proteins^{2,61,64,65}. Supporting this concept, plasma EV surface protein profiling using aptamer panels successfully classified tumor stages, detected lung cancer⁶⁶, and differentiated tumor types⁶⁷. Alternatively, cancer may alter EV release by immune cells through specific crosstalk^{5,68}, aligning with the current observation that pan-cancer-regulated proteins strongly associate with B cell activation, with immunoglobulins IGHV3-13, IGHV4-34, IGKV1-16, IGKV2-28, and IGKV2D-40 showing higher abundance in the pan-cancer setting, and IGKV1-33, IGKV1-17, IGKV3D-11 were differentially expressed in doxorubicin-resistant sarcoma patients. Future studies identifying corona-specific proteins in patient plasma may reveal new insights into EV surface interactions and signaling in health and disease.

EVs exhibit significant heterogeneity in both physical properties and biogenesis pathways, with new subtypes continually being described^{5,42,69,70}. The diverse EV landscape includes multiple varieties of exosomes, exomeres, supermeres, microvesicles, ectosomes, autophagic EVs, migrasomes, apoptotic bodies, matrix vesicles, and exophers, though which specific subtype(s) undergo the most significant alterations in cancer patients remains undetermined.

Relevant to cancer, oncosomes are a subtype of large (1-10 μm) EVs released by prostate cells in vitro⁷⁰, yet size analysis of EVs isolated from sarcoma patients indicates their limited prevalence in this context. Notably, both cancer cohorts demonstrated significant upregulation of mitochondrial proteins, suggesting potential involvement of mitovesicles (or mitoEVs)⁴². Although mitovesicles were initially characterized in brain tissue and have not yet been documented in cancer settings, the well-established connection between altered mitochondrial function and cancer progression warrants further investigation into their potential association and functional significance.

Chemotherapy-induced EVs (ChemoEVs) can significantly influence anticancer treatment sensitivity and tumor behavior⁴⁹. Since doxorubicin targets all proliferating tissues that produce EVs, we expected to observe a clear proteomic signature of treatment. However, comparison of plasma EV proteomes before and after treatment revealed no consistent changes. This could be due to suboptimal post-treatment sample collection timing or because doxorubicin's effect on the plasma EV proteome is heterogeneous, personalized, and lacks a consistent overall pattern. Vinik et al. observed significant changes in dozens of protein expressions in plasma EVs from breast cancer patients after various chemotherapy treatments⁸. However, without pre-treatment samples, that study couldn't assess chemotherapy's direct effect on plasma EVs within individual patients. Notably, it was discovered that the most effective markers of doxorubicin sensitivity were intrinsic and present before treatment—ideal for prediction purposes. Cancer-associated fibroblasts (CAFs) may contribute to chemotherapy-modulated EVs, as He et al. demonstrated that EVs from CAFs isolated from CRC patients promoted chemoresistance in CRC cells in vitro compared to EVs from normal fibroblasts from adjacent tissue⁷¹.

Biomarker discovery efforts have been employed to assess sarcoma and doxorubicin sensitivity. The 67-gene CINSARC signature predicts risk of sarcoma relapse and metastasis, with ongoing clinical trials evaluating its prognostic efficacy for chemotherapy (NCT03805022, NCT04307277)⁷². RNA sequencing of pretreatment tumor samples from the ANNOUNCE trial identified and validated REDSARC, a 25-gene signature predictive of doxorubicin response and survival outcomes in several sarcoma subtypes except undifferentiated pleomorphic sarcoma⁷³. Most sarcoma biomarker studies, including CINSARC, REDSARC, and others^{36,73-75}, are constrained by requiring tissue biopsies, which are more invasive than plasma-and liquid-based biopsies and cannot be obtained serially. Plasma-based sarcoma assays typically employ single biomarker approaches, which have disadvantages compared to the multiparametric biomarker discovery strategies utilized here^{19,76,77}. Machine learning-based classifiers trained on entire proteomes offer advantages but limit clear interpretation, making it difficult to identify which proteins drive classification performance³⁴. Several complementary studies have analyzed the same SARC021 trial data. Tomaszewski et al. combined machine learning with advanced image analysis to identify patients more likely to respond to doxorubicin, revealing a population with significantly better response⁷⁸. Madanat-Harjuoja quantified circulating tumor DNA (ctDNA) in the LMS subset of SARC021 patients, finding that ctDNA detection before or after doxorubicin treatment correlated with significantly lower overall survival⁷⁹. Notably, the plasma EV proteome's diagnostic performance compares favorably with the best predictor of doxorubicin sensitivity in breast cancer patients, which relies on PET/MRI54,⁵⁴.

While over 100 FDA-approved clinical assays target plasma or serum proteins, approval has slowed to merely 2 markers per year, with virtually none focused on drug response classification^{80,81}. Multi-cancer early detection (MECD) tests assaying numerous molecular indicators are now emerging, exemplified by the ambitious FuSion trial (NCT05159544) which will measure 50 molecular indicators across 50,000 individuals⁸². Although this trial will not specifically target EVs or employ proteomics, the integration of advanced proteomic technologies with sophisticated machine learning—as demonstrated in the current study—will revolutionize biomarker discovery by identifying and implementing multi-parameter proteomic classifiers in clinical settings¹⁹. By profiling biospecimens from multicenter trials like SARC021, numerous confounding factors can be eliminated and identify truly robust, portable biomarkers that perform consistently across diverse patient cohorts and clinical sites⁸³. As clinical laboratories increasingly adopt proteomics and achieve CLIA certification, the workflow we report will enable powerful, non-invasive characterization of plasma EVs, transforming our understanding and management of both health and disease.

### The Following Example 8—Materials and Methods

Doxorubicin treatment and plasma collection: Plasma was obtained from anonymized sarcoma patients on the TH CR-406/SARC021 clinical trial, registered with ClinicalTrials.gov as NCT01440088 and entitled “A Trial of TH-302 in Combination With Doxorubicin Versus Doxorubicin Alone to Treat Patients With Locally Advanced Unresectable or Metastatic Soft Tissue Sarcoma”⁸⁴. Patients in SARC021 were treated with doxorubicin alone (75 mg/m²) by either a bolus injection or continuous intravenous infusion based on investigator discretion. Tumor response was assessed at the end of cycle 2 using RECIST version 1.1¹⁸. Blood was collected in EDTA tubes prior to doxorubicin treatment on day 1 and prior to treatment at cycle 3, centrifuged to isolate plasma, and frozen¹⁶. We obtained frozen plasma prior to treatment, or after 2 cycles (6 weeks) of treatment, from the Sarcoma Alliance for Research through Collaboration (SARC) biobank.

Blood was collected from healthy volunteers in EDTA tubes, centrifuged at 3000 rpm for 20 minutes to isolate plasma, aliquoted, and frozen. These anonymized patients were at the Washington University Center for Advanced

Medicine gastrointestinal clinic for a standard of care visit to treat symptoms of gastroesophageal reflux disease. The IRB number for the study is 201111078 and samples were obtained from the Washington University Digestive Diseases Research Core Center Biobank Core. Details of sample preparation for the Hoshino dataset are provided in the original manuscript¹.

EV preparation: EV capture was performed using the METM kit from New England Peptide. Manufacturer instructions were followed with the following modifications. Aliquots of frozen plasma were thawed on ice, vortexed, and spun at 15,000×g for 10 min. 200 μL of plasma was added to a new 1.7 mL microcentrifuge tube [Axygen, Cat. No. MCT-175-C] containing 200 μL of PBS [Gibco™, Cat. No. 370011044] (10X, pH 7.4; diluted to 1× with LC-MS grade water) and mixed by gentle inversion. EV precipitation was initiated by addition of 8 μL Vn96 peptide stock (prepared per manufacturer instructions) followed by mixing via inversion, and incubation at RT with end-over-end rotation for one hour. Samples were centrifuged at 17,000×g for 15 min at 4° C. to collect the EVs at the bottom of the tube. The supernatant was carefully removed, and the pellet was resuspended in 400 μL of PBS. The samples were spun again at 17,000×g for 15 min at 4° C. The EV pellet was washed two more times with PBS as described above. After the last spin, the pellets were stored at −80° C. until peptide preparation. All steps were performed at room temperature unless otherwise noted.

EV size analysis: Dynamic Light Scattering (DLS) was used to analyze size distribution of the plasma EVs. Following precipitation with Vn96 peptide, the pellet was solubilized in PBS/8% trehalose (from 30% w/v trehalose dihydrate stock, Hampton Research) with incubation in a Branson B200 ultrasonic bath for 10 min. Sample was then centrifuged at 14,000×g and analyzed on a Malvern NanoS instrument at 25□C. A representative intensity profile is shown in FIG. 1B. Cumulant size (intensity-weighted Z-average) was reproducible between runs and polydispersity was low which indicated a relatively homogeneous distribution of particles. These results are consistent with measurements from cell-line derived EVs purified by size exclusion chromatography and validated by transmission electron microscopy⁸⁵.

Peptide preparation: Samples were digested as previously described^{86,87} using a modification of the filter-aided sample preparation method⁸⁸. The EV pellets were solubilized with 30 μL SDS buffer (4% (w/v) SDS, 100 mM Tris-HCl pH 8.0). The samples were reduced by addition of 50 mM DTT with heating to 95° C. for 10 min. The reduced samples were mixed with 200 μL of 100 mM Tris-HCL buffer, pH 8.5 containing 8 M urea (UA buffer), transferred to the top chamber of a 30,000 MWCO cutoff filtration unit [Millipore Cat. No. MRCFOR030], and spun in a microcentrifuge at 14,000×g for 10 min. An additional 200 μL of 100 mM Tris-HCL buffer, pH 8.5 containing 8 M urea (UA buffer) was added to the top chamber of the filter unit, and the filter was spun at 14,000×g for 15-20 min in a microcentrifuge [Eppendorf 5424, Eppendorf Cat. No. 2231000767]. The flowthrough was discarded, and the proteins were alkylated by addition of 100 μL of 50 mM lodoacetamide [Pierce, Cat. No. A39271] in UA buffer to the top chamber of the filtration unit and gyrating at 550 rpm in the dark at RT for 30 min using a thermomixer [Eppendorf, Thermomixer R]. The filter was spun at 14,000×g for 15 min, and the flow through discarded. Unreacted iodoacetamide was washed through the filter with two sequential additions of 200 μL of 100 mM Tris-HCL buffer, pH 8.5 containing 8 M urea and centrifuged at 14,000×g for 15-20 min after each addition. The urea buffer was exchanged into digestion buffer (DB), 50 mM ammonium bicarbonate buffer, pH 8. Two sequential additions of DB (200 μL) with centrifugation after each addition to the top chamber was performed. The top filter units were transferred to a new collection tube, 100 μL DB containing 1 μAU of LysC [Wako Chemicals, Cat. No. 129-02541] was added, and samples were digested at 37° C. After 2 hours of LysC digestion, 1 μg of sequencing-grade trypsin [Promega, Cat. No. V5113] was added, and samples were digested overnight at 37° C. The filters were spun at 14,000×g for 15 min to collect the peptides in the flow through. The filter was washed with 50 μL 100 mM ammonium bicarbonate buffer, and the wash was collected with the peptides. In preparation for desalting, peptides were acidified to pH 2 with 1% (vol/vol) TFA. The peptides were desalted using two micro-tips (porous graphite carbon, Glygen BIOMEKNT3CAR) on a Beckman robot

[Biomek NX], as previously described⁸⁹. The peptides were eluted with 60 μL of 60% (vol/vol) acetonitrile in 0.1% TFA (v/v) and dried in a Speed-Vac [Thermo Fisher Scientific, Model No. Savant DNA 120 concentrator] after adding TFA to 5% (v/v). The peptides were dissolved in 20 μL of 1% (v/v) acetonitrile in water. An aliquot (10%) was removed for quantification using the Pierce Quantitative Fluorometric Peptide Assay kit [Thermo Fisher Scientific, Cat. No. 23290]. The remaining peptides were transferred to autosampler vials [Sun-Sri, Cat. No. 200046], dried, and stored at −80° C. for LC-MS analysis. The average peptide yield across samples was 31.5 ug per mL of plasma.

nano-LC-MS/MS: The peptides were analyzed using trapped ion mobility time-of-flight mass spectrometry (PMID3038 5480). Peptides were separated using a nano-ELUTE chromatograph (Bruker Daltonics. Bremen, Germany) interfaced to a timsTOF Pro mass spectrometer (Bruker Daltonics) with a modified nano-electrospray source (CaptiveSpray, Bruker Daltonics). The mass spectrometer was operated in PASEF mode (PMID30385480). The samples in 2 μl of 1% (vol/vol) FA were injected onto a 75 μm i.d.×25 cm Aurora Series column with a CSI emitter (lonopticks). The column temperature was set to 50° C. The column was equilibrated using constant pressure (800 bar) with 8column volumes of solvent A (0.1% (vol/vol) FA). Sample loading was performed at constant pressure (800 bar) at a volume of 1 sample pick-up volume plus 2 μl. The peptides were eluted using one column separation mode with a flow rate of 300 nL/min and using solvents A (0.1% (vol/vol) FA) and B (0.1% (vol/vol) FA/MeCN): solvent A containing 2% B increased to 17% B over 60 min, to 25% B over 30 min, to 37% B over 10 min, to 80% B over 10 min and constant 80% B for 10 min. The MS1 and MS2 spectra were recorded from m/z 100 to 1700.

The collision energy was ramped stepwise as a function of increasing ion mobility: 52 eV for 0-19% of the ramp time; 47 eV from 19-38%; 42 eV from 38-57%; 37 eV from 57-76%; and 32 eV for the remainder. The TIMS elution voltage was calibrated linearly using the Agilent ESI-L Tuning Mix (m/z 622, 922, 1222).

Protein identification: LC-MS data were searched against MaxQuant search engine⁹⁰ (v. 1.6.17.0). MaxQuant was set to search against a concatenated UniProt (May 2020) database of human (20,365 entries) and common contaminant proteins (cRAP, version 1.0 Jan. 1, 2012; 116 entries). Enzyme cleavage specificity was trypsin/P with a maximum of 4 missed cleavages allowed. The MS2 database searches were performed with a fragment ion mass tolerance of 20 ppm and a parent ion tolerance of 20 ppm. Carbamidomethylation of cysteine was specified in MaxQuant as a fixed modification. Deamidation of asparagine, formation of pyro-glutamic acid from N-terminal glutamine, acetylation of protein N-terminus, oxidation of methionine, and pyro-carbamidomethylation of N-terminal cysteine were specified as variable modifications. Peptides and proteins were filtered at 1% false-discovery rate (FDR) by searching against a reversed database. Keratins and abundant plasma proteins were filtered out as potential contaminants, including SERPINA1, HP, IGHA1, IGHG1, FGA, ALB, TF, ORM1, A2M, APOA1, APOA2, C3, IGHM, TTR, and HBD.

Protein expression analysis and statistics: ProteoQ (v1.7.5.1, https://github.com/qzhang503/proteoQ) was used to generate protein expression levels from raw LC-MS using the peptides uniquely assigned to each protein. For log2 normalization, the ratios of gene unique peptides were calculated relative to the MaxQuant LFQ intensities of peptides across all samples, and the medians were taken to represent protein ratios⁹¹. The ratios of peptides and proteins were then transformed such that the median under each sample was zero on a log₂ scale. The offsets used in the median-centering of peptide and protein ratios were applied to scale intensity values accordingly.

Multiple hypotheses correction of p-values was performed using the Benjamini-Hochberg method where indicated. The minimum peptide intensity value in each dataset was used to impute missing values. Harmonization of the sarcoma and Hoshino cohorts was performed with the Harmonizer software package³⁹ after normalization with Box-Cox power transformation. Proteins not detected in at least 15 samples in the pan-cancer dataset were removed prior to downstream analysis of the pan-cancer dataset. PCA and tSNE were formed using scikit-learn. LDA was performed using the MASS⁹² package in R 4.2.1 ported to Python with rpy2 (v3.5.9) using the ‘moment’ method after removing proteins with expression correlations (spearman) greater than 0.67 with other proteins.

Machine learning classification: Samples were randomly assigned to training (60%), validation (15%), and testing (25%) sets prior to any data transformations in order to prevent data leakage. Binning was performed 7 times using random seeds to account for stochastic variation, followed by processing using a scikit-learn-based machine learning pipeline⁹³. Each sample set was independently scaled (RobustScalar, StandardScalar) and 50-100 features were recursively extracted with cross validation (RFECV) using the RepeatedStratifiedKFold cross validator to reduce data dimensionality. 17 machine learning algorithms were then used to analyze the training and validation sets after automated tuning the hyperparameters on the training set using Bayesian optimization, including the HistGradientBoostingClassifier, CatBoostClassifier, LGBMClassifier, XGBClassifier, AdaBoostClassifier, Random ForestClassifier, BaggingClassifier, ExtraTreesClassifier, GradientBoostingClassifier, SVC, MLPClassifier, KNeighborsClassifier, GaussianProcessClassifier, DecisionTreeClassifier, GaussianNB, QuadraticDiscriminantAnalysis, LinearSVC, RidgeClassifier, SGDClassifier, and the XGBClassifier^{93,94}. The performance of each classifier was evaluated by cross validation on the training set scored using the Matthews Correlation Coefficient (MCC) coupled with classification performance (MCC, AUROC) on the heldout Validation set. The best classifier across all 7 randomized iterations was then used to test performance on the heldout Test set.

ROC analysis: Nine proteins which were found differentiating sarcoma tumors from healthy controls were analyzed for ROC analysis to gauge the discriminative abilities of their pre-treatment protein expression for differentiating sarcoma tumors from healthy controls based on area under the ROC curve (AUC). Logistic regression was applied to create an ROC curve and estimate AUC and its 95% CI, using the PROC LOGISTIC procedure in SAS (version 9.4). The estimated AUC of each protein was tested against a null AUC of 0.5 indicating random chance.

ROC analyses was also conducted of all proteins to gauge the discriminative abilities of their pre-treatment protein expression for differentiating sensitive sarcoma tumors from resistant tumors based on area under the ROC curve (AUC). Logistic regression was applied to create an ROC curve and estimate AUC and its 95% CI, using the PROC LOGISTIC procedure in SAS. The estimated AUC of each protein was tested against a null AUC of 0.5 indicating random chance.

Pathway analysis: Over-representation analysis of the plasma EV proteins with significantly altered expression in pan-cancer was performed using the Webgestalt webserver, www.webgestalt.org, with default parameters⁹⁵. The list of all proteins detected served as the reference gene list.

### The Following Example 9—References

1. Hoshino, A., Kim, H.S., Bojmar, L., Gyan, K.E., Cioffi, M., Hernandez, J., Zambirinis, C.P., Rodrigues, G., Molina, H., Heissel, S., et al. (2020). Extracellular Vesicle and Particle Biomarkers Define Multiple Human Cancers. Cell 182, 1044-1061.e18. https://doi.org/10.1016/J.CELL.2020.07.009.
2. Hoshino, A., Costa-Silva, B., Shen, T.-L., Rodrigues, G., Hashimoto, A., Tesic Mark, M., Molina, H., Kohsaka, S., Di Giannatale, A., Ceder, S., et al. (2015). Tumour exosome integrins determine organotropic metastasis. Nature 527, 329-335. https://doi.org/10.1038/nature15756.
3. Luga, V., Zhang, L., Viloria-Petit, A.M., Ogunjimi, A.A., Inanlou, M.R., Chiu, E., Buchanan, M., Hosein, A.N., Basik, M., and Wrana, J.L. (2012). Exosomes mediate stromal mobilization of autocrine Wnt-PCP signaling in breast cancer cell migration. Cell 151, 1542-1556. https://doi.org/10.1016/j.cell.2012.11.024.
4. Peinado, H., Alečković, M., Lavotshkin, S., Matei, I., Costa-Silva, B., Moreno-Bueno, G., Hergueta-Redondo, M., Williams, C., García-Santos, G., Ghajar, C.M., et al. (2012). Melanoma exosomes educate bone marrow progenitor cells toward a pro-metastatic phenotype through MET. Nat Med 18, 883-891. https://doi.org/10.1038/nm.2753.
5. Lucotti, S., Kenific, C.M., Zhang, H., and Lyden, D. (2022). Extracellular vesicles and particles impact the systemic landscape of cancer. EMBO J 41. https://doi.org/10.15252/embj.2021109288.
6. Yang, S., Wang, D., Li, J., Xu, H., Shen, H., Chen, X., Zhou, S., Zhong, S., Zhao, J., and Tang, J. (2017). Predictive role of GSTP1-containing exosomes in chemotherapy-resistant breast cancer. Gene 623, 5-14. https://doi.org/10.1016/j.gene.2017.04.031.
7. Bobrie, A., Krumeich, S., Reyal, F., Recchi, C., Moita, L.F., Seabra, M.C., Ostrowski, M., and Théry, C. (2012). Rab27a Supports Exosome-Dependent and-Independent Mechanisms That Modify the Tumor Microenvironment and Can Promote Tumor Progression. Cancer Res 72, 4920-4930. https://doi.org/10.1158/0008-5472.CAN-12-0925.
8. Vinik, Y., Ortega, F.G., Mills, G.B., Lu, Y., Jurkowicz, M., Halperin,

S., Aharoni, M., Gutman, M., and Lev, S. (2020). Proteomic analysis of circulating extracellular vesicles identifies potential markers of breast cancer progression, recurrence, and response. Sci Adv 6, eaba5714. https://doi.org/10.1126/sciadv.aba5714.
9. Whiteside, T.L. (2020). Validation of plasma-derived small extracellular vesicles as cancer biomarkers. Nature Reviews Clinical Oncology 2020 17:12 17, 719-720. https://doi.org/10.1038/s41571-020-00433-5.
10. Brito-Rocha, T., Constâncio, V., Henrique, R., and Jerónimo, C. (2023). Shifting the Cancer Screening Paradigm: The Rising Potential of Blood-Based Multi-Cancer Early Detection Tests. Cells 12, 935. https://doi.org/10.3390/cells12060935.
11. Mundt, F., Rajput, S., Li, S., Ruggles, K. V., Mooradian, A.D., Mertins, P., Gillette, M.A., Krug, K., Guo, Z., Hoog, J., et al. (2018). Mass Spectrometry-Based Proteomics Reveals Potential Roles of NEK9 and MAP2K4 in Resistance to PI3K Inhibition in Triple-Negative Breast Cancers. Cancer Res 78, 2732-2746. https://doi.org/10.1158/0008-5472.CAN-17-1990.
12. Siegel, R.L., Miller, K.D., Wagle, N.S., and Jemal, A. (2023). Cancer statistics, 2023. CA Cancer J Clin 73, 17-48. https://doi.org/10.3322/caac.21763.
13. Chatterjee, K., Zhang, J., Honbo, N., and Karliner, J.S. (2010). Doxorubicin cardiomyopathy. Cardiology 115, 155-162. https://doi.org/10.1159/000265166.
14. Richards, K.E., Zeleniak, A.E., Fishel, M.L., Wu, J., Littlepage, L.E., and Hill, R. (2017). Cancer-associated fibroblast exosomes regulate survival and proliferation of pancreatic cancer cells. Oncogene 36, 1770-1778. https://doi.org/10.1038/onc.2016.353.
15. Boelens, M.C., Wu, T.J., Nabet, B.Y., Xu, B., Qiu, Y., Yoon, T., Azzam, D.J., Twyman-Saint Victor, C., Wiemann, B.Z., Ishwaran, H., et al. (2014). Exosome Transfer from Stromal to Breast Cancer Cells Regulates Therapy Resistance Pathways. Cell 159, 499-513. https://doi.org/10.1016/j.cell.2014.09.051.
16. Tap, W.D., Papai, Z., Van Tine, B.A., Attia, S., Ganjoo, K.N., Jones, R.L., Schuetze, S., Reed, D., Chawla, S.P., Riedel, R.F., et al. (2017). Doxorubicin plus evofosfamide versus doxorubicin alone in locally advanced, unresectable or metastatic soft-tissue sarcoma (TH CR-406/SARC021): an international, multicentre, open-label, randomised phase 3 trial. Lancet Oncol 18, 1089-1103. https://doi.org/10.1016/S1470-2045(17)30381-9.
17. Barlin, M., Erdmann-Gilmore, P., Mudd, J.L., Zhang, Q., Seymour, R.W., Guo, Z., Miessner, J.R., Goedegebuure, S.P., Bi, Y., Osorio, O.A., et al. (2023). Proteins in Tumor-Derived Plasma Extracellular Vesicles Indicate Tumor Origin. Molecular & Cellular Proteomics 22, 100476. https://doi.org/10.1016/j.mcpro.2022.100476.
18. Eisenhauer, E.A., Therasse, P., Bogaerts, J., Schwartz, L.H., Sargent, D., Ford, R., Dancey, J., Arbuck, S., Gwyther, S., Mooney, M., et al. (2009). New response evaluation criteria in solid tumours: Revised RECIST guideline (version 1.1). Eur J Cancer 45, 228-247. https://doi.org/10.1016/j.ejca.2008.10.026.
19. Geyer, P.E., Holdt, L.M., Teupser, D., and Mann, M. (2017). Revisiting biomarker discovery by plasma proteomics. Mol Syst Biol 13, 942. https://doi.org/10.15252/msb.20156297.
20. Mann, M., Kumar, C., Zeng, W.F., and Strauss, M.T. (2021). Artificial intelligence for proteomics and biomarker discovery. Cell Syst 12, 759-770. https://doi.org/10.1016/J.CELS.2021.06.006.
21. Bijnsdorp, I. v., Maxouri, O., Kardar, A., Schelfhorst, T., Piersma, S.R., Pham, T. v., Vis, A., van Moorselaar, R.J., and Jimenez, C.R. (2017). Feasibility of urinary extracellular vesicle proteome profiling using a robust and simple, clinically applicable isolation method. J Extracell Vesicles 6. https://doi.org/10.1080/20013078.2017.1313091.
22. Joy, A.P., Ayre, D.C., Chute, I.C., Beauregard, A.P., Wajnberg, G., Ghosh, A., Lewis, S.M., Ouellette, R.J., and Barnett, D.A. (2018). Proteome profiling of extracellular vesicles captured with the affinity peptide Vn96: comparison of Laemmli and TRIzol© protein-extraction methods. J Extracell Vesicles 7. https://doi.org/10.1080/20013078.2018.1438727.
23. Ghosh, A., Davey, M., Chute, I.C., Griffiths, S.G., Lewis, S., Chacko, S., Barnett, D., Crapoulet, N., Fournier, S., Joy, A., et al. (2014). Rapid isolation of extracellular vesicles from cell culture and biological fluids using a synthetic peptide with specific affinity for heat shock proteins. PLOS One. https://doi.org/10.1371/journal.pone.0110443.
24. Karimi, N., Cvjetkovic, A., Jang, S.C., Crescitelli, R., Hosseinpour Feizi, M.A., Nieuwland, R., Lötvall, J., and Lässer, C. (2018). Detailed analysis of the plasma extracellular vesicle proteome after separation from lipoproteins. Cellular and Molecular Life Sciences 75, 2873-2886. https://doi.org/10.1007/s00018-018-2773-4.
25. Bijnsdorp, I. V., Maxouri, O., Kardar, A., Schelfhorst, T., Piersma, S.R., Pham, T. V., Vis, A., Van Moorselaar, R.J., and Jimenez, C.R. (2017). Feasibility of urinary extracellular vesicle proteome profiling using a robust and simple, clinically applicable isolation method. J Extracell Vesicles 6. https://doi.org/10.1080/20013078.2017.1313091.
26. Jeppesen, D.K., Fenix, A.M., Franklin, J.L., Higginbotham, J.N., Zhang, Q., Zimmerman, L.J., Liebler, D.C., Ping, J., Liu, Q., Evans, R., et al. (2019). Reassessment of Exosome Composition. Cell 177, 428-445.e18. https://doi.org/10.1016/j.cell.2019.02.029.
27. Zhang, H., Freitas, D., Kim, H.S., Fabijanic, K., Li, Z., Chen, H., Mark, M.T., Molina, H., Martin, A.B., Bojmar, L., et al. (2018). Identification of distinct nanoparticles and subsets of extracellular vesicles by asymmetric flow field-flow fractionation. Nat Cell Biol 20, 332-343. https://doi.org/10.1038/s41556-018-0040-4.
28. Keerthikumar, S., Chisanga, D., Ariyaratne, D., Al Saffar, H., Anand, S., Zhao, K., Samuel, M., Pathan, M., Jois, M., Chilamkurti, N., et al. (2016). ExoCarta: A Web-Based Compendium of Exosomal Cargo. J Mol Biol 428, 688-692.
29. Mathivanan, S., Lim, J.W.E., Tauro, B.J., Ji, H., Moritz, R.L., and Simpson, R.J. (2010). Proteomics Analysis of A33 Immunoaffinity-purified

Exosomes Released from the Human Colon Tumor Cell Line LIM1215 Reveals a Tissue-specific Protein Signature. Molecular & Cellular Proteomics 9, 197-208. https://doi.org/10.1074/mcp.M900152-MCP200.
30. Looze, C., Yui, D., Leung, L., Ingham, M., Kaler, M., Yao, X., Wu, W.W., Shen, R.-F., Daniels, M.P., and Levine, S.J. (2009). Proteomic profiling of human plasma exosomes identifies PPARy as an exosome-associated protein. Biochem Biophys Res Commun 378, 433-438. https://doi.org/10.1016/j.bbrc.2008.11.050.
31. Kharaziha, P., Chioureas, D., Rutishauser, D., Baltatzis, G., Lennartsson, L., Fonseca, P., Azimi, A., Hultenby, K., Zubarev, R., Ullén, A., et al. (2015). Molecular profiling of prostate cancer derived exosomes may reveal a predictive signature for response to docetaxel. Oncotarget 6, 21740-21754. https://doi.org/10.18632/oncotarget.3226.
32. Demory Beckler, M., Higginbotham, J.N., Franklin, J.L., Ham, A.-J., Halvey, P.J., Imasuen, I.E., Whitwell, C., Li, M., Liebler, D.C., and Coffey, R.J. (2013). Proteomic Analysis of Exosomes from Mutant KRAS Colon Cancer Cells Identifies Intercellular Transfer of Mutant KRAS. Molecular & Cellular Proteomics 12, 343-355. https://doi.org/10.1074/mcp.M112.022806.
33. Liang, B., Peng, P., Chen, S., Li, L., Zhang, M., Cao, D., Yang, J., Li, H., Gui, T., Li, X., et al. (2013). Characterization and proteomic analysis of ovarian cancer-derived exosomes. J Proteomics 80, 171-182. https://doi.org/10.1016/j.jprot.2012.12.029.
34. Pedregosa, F., Varoquaux, G., Gramfort, A., Michel, V., Thirion, B., Grisel, O., Blondel, M., Prettenhofer, P., Weiss, R., Dubourg, V., et al. (2011). Scikit-learn: Machine Learning in Python. Journal of Machine Learning Research 12, 2825-2830.
35. Vabalas, A., Gowen, E., Poliakoff, E., and Casson, A.J. (2019). Machine learning algorithm validation with a limited sample size. PLOS One 14, 1-20. https://doi.org/10.1371/journal.pone.0224365.
36. Taylor, B.S., Barretina, J., Maki, R.G., Antonescu, C.R., Singer, S., and Ladanyi, M. (2011). Advances in sarcoma genomics and new therapeutic targets. Nature Reviews Cancer 2011 11:8 11, 541-557. https://doi.org/10.1038/nrc3087.
37. Abeshouse, A., Adebamowo, C., Adebamowo, S.N., Akbani, R., Akeredolu, T., Ally, A., Anderson, M.L., Anur, P., Appelbaum, E.L., Armenia, J., et al. Comprehensive and Integrated Genomic Characterization of Adult Soft Tissue Sarcomas. Cell 171, 950-965.e28. https://doi.org/10.1016/j.cell.2017.10.014.
38. Nacev, B.A., Sanchez-Vega, F., Smith, S.A., Antonescu, C.R., Rosenbaum, E., Shi, H., Tang, C., Socci, N.D., Rana, S., Gularte-Mérida, R., et al. (2022). Clinical sequencing of soft tissue and bone sarcomas delineates diverse genomic landscapes and potential therapeutic targets. Nat Commun 13, 3405. https://doi.org/10.1038/s41467-022-30453-x.
39. Voß, H., Schlumbohm, S., Barwikowski, P., Wurlitzer, M., Dottermusch, M., Neumann, P., Schlüter, H., Neumann, J.E., and Krisp, C. (2022). HarmonizR enables data harmonization across independent proteomic datasets with appropriate handling of missing values. Nature Communications 2022 13:1 13, 1-15. https://doi.org/10.1038/s41467-022-31007-x.
40. The UniProt Consortium (2019). UniProt: a worldwide hub of protein knowledge. Nucleic Acids Res 47, D506-D515. https://doi.org/10.1093/nar/gky1049.
41. Towers, C.G., Wodetzki, D.K., Thorburn, J., Smith, K.R., Caino, M.C., and Thorburn, A. (2021). Mitochondrial-derived vesicles compensate for loss of LC3-mediated mitophagy. Dev Cell 56, 2029-2042.e5. https://doi.org/10.1016/j.devcel.2021.06.003.
42. D′Acunzo, P., Pérez-González, R., Kim, Y., Hargash, T., Miller, C., Alldred, M.J., Erdjument-Bromage, H., Penikalapati, S.C., Pawlik, M., Saito, M., et al. (2021). Mitovesicles are a novel population of extracellular vesicles of mitochondrial origin altered in Down syndrome. Sci Adv 7. https://doi.org/10.1126/sciadv.abe5085.
43. Hanahan, D., and Weinberg, R.A. (2011). Hallmarks of cancer: the next generation. Cell 144, 646-674. https://doi.org/10.1016/j.cell.2011.02.013.
44. Zhang, J., Shi, J., Zhang, H., Zhu, Y., Liu, W., Zhang, K., and Zhang, Z. (2020). Localized fluorescent imaging of multiple proteins on individual extracellular vesicles using rolling circle amplification for cancer diagnosis. J Extracell Vesicles 10. https://doi.org/10.1002/jev2.12025.
45. Liu, C., Zhao, J., Tian, F., Cai, L., Zhang, W., Feng, Q., Chang, J., Wan, F., Yang, Y., Dai, B., et al. (2019). Low-cost thermophoretic profiling of extracellular-vesicle surface proteins for the early detection and classification of cancers. Nat Biomed Eng 3, 183-193. https://doi.org/10.1038/s41551-018-0343-6.
46. Hinestrosa, J.P., Kurzrock, R., Lewis, J.M., Schork, N.J., Schroeder, G., Kamat, A.M., Lowy, A.M., Eskander, R.N., Perrera, O., Searson, D., et al. (2022). Early-stage multi-cancer detection using an extracellular vesicle protein-based blood test. Communications Medicine 2, 29. https://doi.org/10.1038/s43856-022-00088-6.
47. Rassy, E., and Pavlidis, N. (2019). The currently declining incidence of cancer of unknown primary. Cancer Epidemiol 61, 139-141. https://doi.org/10.1016/J.CANEP.2019.06.006.
48. Gupta, A., Sagar, G., Siddiqui, Z., Rao, K.V.S., Nayak, S., Saquib, N., and Anand, R. (2022). A non-invasive method for concurrent detection of early-stage women-specific cancers. Sci Rep 12, 2301. https://doi.org/10.1038/s41598-022-06274-9.
49. Ab Razak, N.S., Ab Mutalib, N.S., Mohtar, M.A., and Abu, N. (2019). Impact of Chemotherapy on Extracellular Vesicles: Understanding the Chemo-EVs. Front Oncol 9, 1113. https://doi.org/10.3389/FONC.2019.01113/BIBTEX.
50. Chen, W.X., Liu, X.M., Lv, M.M., Chen, L., Zhao, J.H., Zhong, S.L., Ji, M.H., Hu, Q., Luo, Z., Wu, J.Z., et al. (2014). Exosomes from Drug-Resistant Breast Cancer Cells Transmit Chemoresistance by a Horizontal Transfer of MicroRNAs. PLOS One 9. https://doi.org/10.1371/JOURNAL.PONE.0095240.
51. Su, Y., Li, Y., Guo, R., Zhao, J., Chi, W., Lai, H., Wang, J., Wang, Z., Li, L., Sang, Y., et al. (2021). Plasma extracellular vesicle long RNA profiles in the diagnosis and prediction of treatment response for breast cancer. npj Breast Cancer 2021 7:1 7, 1-10. https://doi.org/10.1038/s41523-021-00356-z.
52. Beaumier, A., Robinson, S.R., Robinson, N., Lopez, K.E., Meola, D.M., Barber, L.G., Bulmer, B.J., Calvalido, J., Rush, J.E., Yeri, A., et al. (2020). Extracellular vesicular microRNAs as potential biomarker for early detection of doxorubicin-induced cardiotoxicity. J Vet Intern Med 34, 1260. https://doi.org/10.1111/JVIM.15762.
53. Chen, T., and Guestrin, C. (2016). XGBoost: A Scalable Tree Boosting System. In Proceedings of the 22nd ACM SIGKDD International Conference on Knowledge Discovery and Data Mining (ACM), pp. 785-794. https://doi.org/10.1145/2939672.2939785.
54. Choi, J.H., Kim, H.-A., Kim, W., Lim, I., Lee, I., Byun, B.H., Noh, W.C., Seong, M.-K., Lee, S.-S., Kim, B. Il, et al. (2020). Early prediction of neoadjuvant chemotherapy response for advanced breast cancer using PET/MRI image deep learning. Sci Rep 10, 21149. https://doi.org/10.1038/s41598-020-77875-5.
55. Chatterjee, K., Zhang, J., Honbo, N., and Karliner, J.S. (2010). Doxorubicin Cardiomyopathy. Cardiology 115, 155-162. https://doi.org/10.1159/000265166.
56. Johnson-Arbor, K., and Dubey, R. (2023). Doxorubicin.
57. Ris, F., Hellan, M., Douissard, J., Nieva, J.J., Triponez, F., Woo, Y., Geller, D., Buchs, N.C., Buehler, L., Moenig, S., et al. (2021). Blood-Based Multi-Cancer Detection Using a Novel Variant Calling Assay (DEEPGEN™): Early Clinical Results. Cancers (Basel) 13, 4104. https://doi.org/10.3390/cancers13164104.
58. Cohen, J.D., Li, L., Wang, Y., Thoburn, C., Afsari, B., Danilova, L., Douville, C., Javed, A.A., Wong, F., Mattox, A., et al. (2018). Detection and localization of surgically resectable cancers with a multi-analyte blood test. Science (1979) 359, 926-930. https://doi.org/10.1126/science.aar3247.
59. Phallen, J., Sausen, M., Adleff, V., Leal, A., Hruban, C., White, J., Anagnostou, V., Fiksel, J., Cristiano, S., Papp, E., et al. (2017). Direct detection of early-stage cancers using circulating tumor DNA. Sci Transl Med 9. https://doi.org/10.1126/scitranslmed.aan2415.
60. Xu, R., Rai, A., Chen, M., Suwakulsiri, W., Greening, D.W., and Simpson, R.J. (2018). Extracellular vesicles in cancer—implications for future improvements in cancer care. Preprint, https://doi.org/10.1038/s41571-018-0036-9 https://doi.org/10.1038/s41571-018-0036-9.
61. Rai, A., Fang, H., Claridge, B., Simpson, R.J., and Greening, D.W. (2021). Proteomic dissection of large extracellular vesicle surfaceome unravels interactive surface platform. J Extracell Vesicles 10, e12164. https://doi.org/10.1002/JEV2.12164.
62. Meng, Q., Chen, C., Yang, N., Gololobova, O., Shi, C., Dunn, C.A., Rossi, M., Martindale, J.L., Basisty, N., Ding, J., et al. (2023). Surfaceome analysis of extracellular vesicles from senescent cells uncovers uptake repressor DPP4. Proceedings of the National Academy of Sciences 120, e2219801120. https://doi.org/10.1073/PNAS.2219801120.
63. König, L., Kasimir-Bauer, S., Bittner, A.-K., Hoffmann, O., Wagner, B., Santos Manvailer, L.F., Kimmig, R., Horn, P.A., and Rebmann, V. (2018). Elevated levels of extracellular vesicles are associated with therapy failure and disease progression in breast cancer patients undergoing neoadjuvant chemotherapy. Oncoimmunology 7, e1376153. https://doi.org/10.1080/2162402X.2017.1376153.
64. Couto, N., Elzanowska, J., Maia, J., Batista, S., Pereira, C.E., Beck, H.C., Carvalho, A.S., Strano Moraes, M.C., Carvalho, C., Oliveira, M., et al. (2022). IgG+ Extracellular Vesicles Measure Therapeutic Response in Advanced Pancreatic Cancer. Cells 11, 2800. https://doi.org/10.3390/CELLS11182800/S1.
65. Castillo, J., Bernard, V., San Lucas, F.A., Allenson, K., Capello, M., Kim, D.U., Gascoyne, P., Mulu, F.C., Stephens, B.M., Huang, J., et al. (2018). Surfaceome profiling enables isolation of cancer-specific exosomal cargo in liquid biopsies from pancreatic cancer patients. Annals of Oncology 29, 223-229. https://doi.org/10.1093/annonc/mdx542.
66. Blume, J.E., Manning, W.C., Troiano, G., Hornburg, D., Figa, M., Hesterberg, L., Platt, T.L., Zhao, X., Cuaresma, R.A., Everley, P.A., et al. (2020). Rapid, deep and precise profiling of the plasma proteome with multi-nanoparticle protein corona. Nat Commun 11, 3662. https://doi.org/10.1038/s41467-020-17033-7.
67. Liu, C., Zhao, J., Tian, F., Cai, L., Zhang, W., Feng, Q., Chang, J., Wan, F., Yang, Y., Dai, B., et al. (2019). Low-cost thermophoretic profiling of extracellular-vesicle surface proteins for the early detection and classification of cancers. Nat Biomed Eng 3, 183-193. https://doi.org/10.1038/s41551-018-0343-6.
68. Wang, X., Tian, L., Lu, J., and Ng, I.O.-L. (2022). Exosomes and cancer—Diagnostic and prognostic biomarkers and therapeutic vehicle. Oncogenesis 11, 54. https://doi.org/10.1038/s41389-022-00431-5.
69. Jeppesen, D.K., Zhang, Q., Franklin, J.L., and Coffey, R.J. (2023). Extracellular vesicles and nanoparticles: emerging complexities. Trends Cell Biol 33, 667-681. https://doi.org/10.1016/j.tcb.2023.01.002.
70. Minciacchi, V.R., Spinelli, C., Reis-Sobreiro, M., Cavallini, L., You, S., Zandian, M., Li, X., Mishra, R., Chiarugi, P., Adam, R.M., et al. (2017). MYC Mediates Large Oncosome-Induced Fibroblast Reprogramming in Prostate Cancer. Cancer Res 77, 2306-2317. https://doi.org/10.1158/0008-5472.CAN-16-2942.
71. Hu, J.L., Wang, W., Lan, X.L., Zeng, Z.C., Liang, Y.S., Yan, Y.R., Song, F.Y., Wang, F.F., Zhu, X.H., Liao, W.J., et al. (2019). CAFs secreted exosomes promote metastasis and chemotherapy resistance by enhancing cell stemness and epithelial-mesenchymal transition in colorectal cancer. Mol Cancer 18, 91. https://doi.org/10.1186/s12943-019-1019-x.
72. Chibon, F., Lagarde, P., Salas, S., Perot, G., Brouste, V., Tirode, F., Lucchesi, C., de Reynies, A., Kauffmann, A., Bui, B., et al. Validated prediction of clinical outcome in sarcomas and multiple types of cancer on the basis of a gene expression signature related to genome complexity. Nat Med 16, 781-787. https://doi.org/nm.2174 [pii] 10.1038/nm.2174.
73. Liu, J., Moura, D.S., Jones, R.L., Aggarwal, A., Ebert, P.J., Wagner, A.J., Wright, J., Martin-Broto, J., and Tap, W.D. (2024). Best Overall Response—Associated Signature to Doxorubicin in Soft Tissue Sarcomas: A Transcriptomic Analysis from ANNOUNCE. Clinical Cancer Research 30, 2598-2608. https://doi.org/10.1158/1078-0432.CCR-23-3936.
74. Suehara, Y., Kubota, D., Kikuta, K., Kaneko, K., Kawai, A., and Kondo, T. (2012). Discovery of biomarkers for osteosarcoma by proteomics approaches. Sarcoma 2012. https://doi.org/10.1155/2012/425636.
75. Milighetti, M., Krasny, L., Lee, A.T.J., Morani, G., Szecsei, C., Chen, Y., Guljar, N., McCarthy, F., Wilding, C.P., Arthur, A., et al. (2021). Proteomic profiling of soft tissue sarcomas with SWATH mass spectrometry. J Proteomics 241, 104236. https://doi.org/10.1016/j.jprot.2021.104236.
76. Wang, J., Guo, W., Wang, X., Tang, X., Sun, X., and Ren, T. (2022). Circulating Exosomal PD-L1 at Initial Diagnosis Predicts Outcome and Survival of Patients with Osteosarcoma. Clinical Cancer Research, OF1-OF8. https://doi.org/10.1158/1078-0432.CCR-22-2682.
77. Fahs, A., Hussein, N., Zalzali, H., Ramadan, F., Ghamloush, F., Tamim, H., El Homsi, M., Badran, B., Boulos, F., Tawil, A., et al. (2022). CD147 Promotes Tumorigenesis via Exosome-Mediated Signaling in Rhabdomyosarcoma. Cells 11. https://doi.org/10.3390/cells11152267.
78. Tomaszewski, M.R., Fan, S., Garcia, A., Qi, J., Kim, Y., Gatenby, R.A., Schabath, M.B., Tap, W.D., Reinke, D.K., Makanji, R.J., et al. (2022). AI-Radiomics Can Improve Inclusion Criteria and Clinical Trial Performance. Tomography 8, 341-355. https://doi.org/10.3390/tomography8010028.
79. Madanat-Harjuoja, L.M., Klega, K., Lu, Y., Shulman, D.S., Thorner, A.R., Nag, A., Tap, W.D., Reinke, D.K., Diller, L., Ballman, K. V., et al. (2022). Circulating Tumor DNA Is Associated with Response and Survival in Patients with Advanced Leiomyosarcoma. Clinical Cancer Research 28, 2579-2586. https://doi.org/10.1158/1078-0432.CCR-21-3951.
80. Biomarkers—Early Detection Research Network https://edrn.nci.nih.gov/data-and-resources/biomarkers/.
81. Bader, J.M., Albrecht, V., and Mann, M. (2023). MS-based proteomics of body fluids: The end of the beginning. Molecular & Cellular Proteomics, 100577. https://doi.org/10.1016/J.MCPRO.2023.100577.
82. Suo, C., Zhao, R., Jiang, Y., Zhang, Y., He, Q., Su, Z., Liu, R., Jin, L., and Chen, X. (2023). Abstract 4194: The FuSion Project of Pan-Cancer Early Screening in Chinese—An integrative study by Fudan University and Singlera. Cancer Res 83, 4194-4194. https://doi.org/10.1158/1538-7445.AM2023-4194.
83. Zhang, Z., and Chan, D.W. (2010). The road from discovery to clinical diagnostics: Lessons learned from the first FDA-cleared in vitro diagnostic multivariate index assay of proteomic biomarkers. Cancer Epidemiology Biomarkers and Prevention 19, 2995-2999. https://doi.org/10.1158/1055-9965.EPI-10-0580/66962/AM/THE-ROAD-FROM-DISCOVERY-TO-CLINICAL-DIAGNOSTICS.
84. Tap, W.D., Papai, Z., Van Tine, B.A., Attia, S., Ganjoo, K.N., Jones, R.L., Schuetze, S., Reed, D., Chawla, S.P., Riedel, R.F., et al. (2017). Doxorubicin plus evofosfamide versus doxorubicin alone in locally advanced, unresectable or metastatic soft-tissue sarcoma (TH CR-406/SARC021): an international, multicentre, open-label, randomised phase 3 trial. Lancet Oncol 18, 1089-1103. https://doi.org/10.1016/S1470-2045(17)30381-9.
85. Katz-Kiriakos, E., Steinberg, D.F., Kluender, C.E., Osorio, O.A., Newsom-Stewart, C., Baronia, A., Byers, D.E., Holtzman, M.J., Katafiasz, D., Bailey, K.L., et al. (2021). Epithelial IL-33 appropriates exosome trafficking for secretion in chronic airway disease. JCI Insight 6. https://doi.org/10.1172/jci.insight.136166.
86. Erde, J., Loo, R.R.O., and Loo, J.A. (2017). Improving Proteome Coverage and Sample Recovery with Enhanced FASP (eFASP) for Quantitative Proteomic Experiments. Methods in Molecular Biology 1550, 11-18. https://doi.org/10.1007/978-1-4939-6747-6_2.
87. Kim, D.I., Jensen, S.C., Noble, K.A., Kc, B., Roux, K.H., Motamedchaboki, K., and Roux, K.J. (2016). An improved smaller biotin ligase for BioID proximity labeling. Mol Biol Cell 27, 1188-1196. https://doi.org/10.1091/mbc.E15-12-0844.
88. Wiśniewski, J.R., Zougman, A., Nagaraj, N., and Mann, M. (2009). Universal sample preparation method for proteome analysis. Nat Methods 6, 359-362. https://doi.org/10.1038/nmeth.1322.
89. Chen, Z.W., Fuchs, K., Sieghart, W., Townsend, R.R., and Evers, A.S. (2012). Deep amino acid sequencing of native brain GABAA receptors using high-resolution mass spectrometry. Molecular and Cellular Proteomics 11, 1-18. https://doi.org/10.1074/mcp.M111.011445.
90. Cox, J., and Mann, M. (2008). MaxQuant enables high peptide identification rates, individualized p.p.b.-range mass accuracies and proteome-wide protein quantification. Nat Biotechnol 26, 1367-1372. https://doi.org/10.1038/nbt. 1511.
91. Cox, J., Hein, M.Y., Luber, C.A., Paron, I., Nagaraj, N., and Mann, M. (2014). Accurate proteome-wide label-free quantification by delayed normalization and maximal peptide ratio extraction, termed MaxLFQ. Molecular and Cellular Proteomics 13, 2513-2526. https://doi.org/10.1074/mcp.M113.031591.
92. Venables, W., and Rupley, B. (2002). Modern Applied Statistics with S Fourth. (Springer).
93. Buitinck, L., Louppe, G., Blondel, M., Pedregosa, F., Mueller, A., Grisel, O., Niculae, V., Prettenhofer, P., Gramfort, A., Grobler, J., et al. (2013). API design for machine learning software: experiences from the scikit-learn project.
94. Akiba, T., Sano, S., Yanase, T., Ohta, T., and Koyama, M. (2019). Optuna: A Next-generation Hyperparameter Optimization Framework.
95. Liao, Y., Wang, J., Jaehnig, E.J., Shi, Z., and Zhang, B. (2019). WebGestalt 2019: gene set analysis toolkit with revamped Uls and APIs. Nucleic Acids Res 47, W199-W205. https://doi.org/10.1093/NAR/GKZ401.

What is claimed is:

1. A method for identifying a human subject as a candidate for treating cancer, the method comprising:
determining whether or not a sample from the subject comprises:
i) a plasma extracellular vesicle; and/or
ii) a plasma extracellular vesicle protein; and
identifying the subject as a candidate for treating cancer by administering an anticancer treatment wherein the anticancer treatment is not administering doxorubicin when the plasma extracellular vesicle and/or the plasma extracellular vesicle protein is detected.

2. The method of claim 1, wherein the plasma extracellular vesicle protein is selected from the group consisting of phosphorylase b kinase regulatory subunit alpha, liver isoform (PHKA2), integrin alpha-5 (ITGA5), sphingolipid delta(4)-desaturase DES (DEGS1), E3 ubiquitin/ISG15 ligase (TRIM25), conserved oligomeric Golgi complex subunit 3 (COG3), rab3 GTPase-activating protein non-catalytic subunit (RAB3GAP2), mannosyl-oligosaccharide glucosidase (MOGS), ER membrane protein complex subunit 10 (EMC10), bifunctional purine biosynthesis protein (ATIC), ribose-5-phosphate isomerase (RPIA), sterol-4-alpha-carboxylate 3-dehydrogenase, decarboxylating (NSDHL), dehydrogenase/reductase SDR family member 7B (DHRS7B), LIM domain-containing protein 1 (LIMD1), thymidine kinase 2, mitochondrial (TK2), isovaleryl-CoA dehydrogenase, mitochondrial (IVD), NIF3-like protein 1 (NIF3L1), ubiquitin carboxyl-terminal hydrolase 15 (USP15), asparagine—tRNA ligase, cytoplasmic (NARS1), immunoglobulin lambda variable 2-11 (IGLV2-11), semenogelin-1 (SEMG1), immunoglobulin kappa variable 1-13 (IGKV1-13), acylamino-acid-releasing enzyme (APEH), aldo-keto reductase family 1 member A1 (AKR1A1), sodium/potassium-transporting ATPase subunit beta-3 (ATP1B3), F-actin-uncapping protein LRRC16A (CARMIL1), nuclear migration protein (NUDC), sorting nexin-2 (SNX2), immunoglobulin lambda variable 4-69 (IGLV4-69), phosphatidylinositol 4-phosphate 5-kinase type-1 gamma (PIP5K1C), supervillain (SVIL), prelamin-A/C (LMNA), and combinations thereof.

3. The method of claim 2, wherein the plasma extracellular vesicle protein is phosphorylase b kinase regulatory subunit alpha (PHKA2).

4. The method of claim 2, wherein the plasma extracellular vesicle protein is sphingolipid delta(4)-desaturase DES (DEGS1).

5. The method of claim 1, wherein the cancer is selected from the group consisting of lung cancer, prostate cancer, breast cancer, liver cancer, pancreatic cancer, kidney cancer, colon cancer, ovarian cancer, skin cancer, or a sarcoma.

6. The method of claim 5, the sarcoma is selected from the group consisting of angiosarcoma, epitheloid sarcoma, leiomyosarcoma, liposarcoma, myxofibrosarcoma, osteosarcoma, and undifferentiated pleomorphic sarcoma/malignant fibrous histiocytoma.

7. The method of claim 5, wherein the cancer is resistant to doxorubicin.

8. The method of claim 5, wherein the cancer is sensitive to doxorubicin.

9. The method of claim 1, wherein the sample from the subject is selected from the group consisting of whole blood, blood serum, blood plasma, urine, saliva, sputum, breast milk, ascites fluid, synovial fluid, amniotic fluid, semen, cerebrospinal fluid, follicular fluid and tears.

10. The method of claim 9, wherein the sample from the subject is a whole blood.

11. The method of claim 1, wherein the subject is a healthy subject.

12. The method of claim 1, wherein the subject is undergoing treatment for cancer.

13. The method of claim 1, wherein the subject is in remission for cancer.

14. The method of claim 1, wherein the anticancer treatment is selected from the group consisting of chemotherapy, hormone therapy, targeted therapeutic therapy, immunotherapy, and combinations thereof.

15. The method of claim 14, wherein the chemotherapy is selected from the group consisting of anthracyclines, taxanes, and platinum agents.

16. The method of claim 14, wherein the hormone therapy is selected from the group consisting of tamoxifen, toremifene, fulvestrant, letrozole, anastrozole, and exemestane.

17. The method of claim 14, wherein the targeted therapeutic therapy is selected from the group consisting of monoclonal antibody therapeutics, antibody-drug conjugates, kinase inhibitors, CDK4/6 inhibitors, mTOR inhibitors, PI3K inhibitors, and PARP inhibitors.

18. The method of claim 14, wherein the immunotherapy is selected from the group consisting of immune checkpoint inhibitors and PD-1 inhibitors.

19. A method for treating a human subject having cancer, the method comprising:
performing or having performed an assay on a biological sample from the subject to identify if the subject as having:
i) a plasma extracellular vesicle; and/or
ii) a plasma extracellular vesicle protein; and
administering an anticancer treatment wherein the anticancer treatment is not administering doxorubicin to the subject having the plasma extracellular vesicle and/or the plasma extracellular vesicle protein;
wherein the presence of the plasma extracellular vesicle and/or the plasma extracellular vesicle protein indicates that the subject is a candidate for treating the cancer by administering an anticancer treatment wherein the anticancer treatment is not administering doxorubicin.

20. A kit comprising:
a plasma extracellular vesicle;
a plasma extracellular vesicle protein;
a substrate for the plasma extracellular vesicle;
a substrate for the plasma extracellular vesicle protein; and
instructions for using the plasma extracellular vesicle and substrate and/or the plasma extracellular vesicle protein and substrate in a method for screening a test subject for cancer treatment.
